(19) United States
(12) Patent Application Publication
(10) Pub. No.: US 2021/0215726 A1
(43) Pub. Date: Jul. 15, 2021

(54) METHOD FOR ASSAYING D-DIMERS SPECIFIC TO VENOUS THROMBOEMBOLISM AND USE THEREOF FOR DIAGNOSING PULMONARY EMBOLISM AND DEEP VENOUS THROMBOSIS

(71) Applicant: Diagnostica Stago, Asnieres S/seine (FR)

(72) Inventors: Genevieve Contant, Courbevoie (FR); Francoise Beaupere, Eaubonne (FR)

(21) Appl. No.: 15/999,798

(22) PCT Filed: Feb. 17, 2017
(86) PCT No.: PCT/FR2017/050354

§ 371 Date: Aug. 17, 2018

(30) Foreign Application Priority Data
Feb. 18, 2016 (FR) 16 51348

(51) Int. Cl. G01N 33/86; C12Q 1/56

(52) U.S. Cl. CPC G01N 33/86; G01N 2800/226; C12Q 1/56

(57) ABSTRACT

The present invention relates to a method for assaying D-dimers that are specific to venous thromboembolism, resulting from the degradation of intravascular fibrin in a blood sample, including routine assay of D-dimers contained in the sample and dynamic measurement of fibrin formation in same. This method may be used to rule out pulmonary embolism or deep venous thrombosis in patients and/or to diagnose thrombosis or coagulation activation or thrombophilia in patients.

### PARENT APPLICATION

The present international application claims the priority of French application number FR 16 51348 filed on Feb. 18, 2016, the content of which is incorporated herein by reference in its entirety.

### FIELD OF THE INVENTION

The present invention relates to a method for assaying D-dimers specific for venous thromboembolism in a blood sample, comprising, on the one hand, the routine assaying of D-dimers of the sample and, on the other hand, the dynamic measurement of fibrin formation of this sample. The method is intended in particular for the in vitro diagnosis of pulmonary embolism and deep vein thrombosis in a blood sample.

### CONTEXT OF THE INVENTION

Venous thromboembolism (VTE) is a major public health problem. It groups together the notion of deep vein thrombosis (DVT) and its immediate vital risk, pulmonary embolism (PE). The basic principles of the pathogenesis of VTE have been described by Virchow who defined the origin of thrombosis in the combination of three thrombosis-promoting factors, which are venous stasis; dysfunction or impairment of the endothelium; and hypercoagulability due to the activation of coagulation factors, to hyperviscosity, to an antithrombin deficiency, to thrombophilia, to nephrotic syndrome, to a change after serious physical trauma or burns, a disseminated cancer, a late pregnancy, ethnicity, age, smoking or obesity (Lopez, Thromb. Res., 2009, 123(Suppl. 4): S30-34).

Pulmonary embolism (PE) is a disease feared by all emergency physicians because it is difficult to diagnose, perhaps one of the most difficult, since it so often manifests itself with very different symptoms, or even is complete asymptomatic. Pulmonary embolism is such a common condition that any respiratory abnormality which occurs abruptly should suggest this diagnosis. It causes acute and scary chest pain. It must be differentiated from myocardial infarction by means of an electrocardiogram, the assaying of cardiac enzymes (transaminases, troponin, etc.) and pulmonary scintigraphy. It must be differentiated from acute pericarditis and from aortic dissection, which contraindicate the use of anticoagulants.

Several types of PE exist:
pulmonary embolism with acute cor pulmonale can be deadly and occurs especially in a patient who is bedridden following a surgical procedure. It may worsen over the first hours, requiring a surgical procedure or embolectomy or thrombolytic treatment. A fatal outcome remains very common;
violent pulmonary embolisms causing sudden death or death in a few minutes by collapse;
pulmonary embolisms which are not very apparent are very common, with symptoms that are often misleading. The diagnosis is often arrived at a few days later; and
pulmonary embolisms in patients suffering from cardiac and respiratory insufficiency are common and their seriousness requires preventive anticoagulant treatment.

The clinical signs of deep vein thrombosis (DVT) are often not very apparent or even non-existent, or similar to those of a superficial vein thrombosis, which is much less serious since it does not progress to PE. It is necessary to differentiate DVT:
from a deep hematoma, since anticoagulant treatment will promote bleeding,
from a subcutaneous infectious disease of the leg,
from a postphlebitic disease, and
from the rupture of a popliteal cyst which gives quite a similar clinical picture.
   There are three types of complications, which are: PE, which, in close to half the cases, is completely asymptomatic; extension of the thrombus in the venous system; and postphlebitic disease, a common complication, in close to a third of patients.

Scores make it possible to evaluate the clinical probability of a PE in the presence of dyspnea or of chest pain. They are used in the diagnostic decision tree to optimize patient treatment, in particular to decide on the therapeutic approach. The current diagnostic algorithm is based on an approach which comprises:
a first step of evaluating the clinical probability: Wells score in DVT (Wells, J. Thromb. Haemost., 2007, 5(Suppl. 1): 41-50), Wells score or Genève score and their simplified versions in PE (Ceriani, J. Thromb. Haemost., 2010, 8(5): 957-970; Klok, Arch. Intern. Med., 2008, 168(19): 2131-2136; Le Gal, Ann. Intern. Med., 2006, 144(3): 165-171), grouping together several risk factors, symptoms and clinical signs,
followed in the majority of cases by assaying of D-dimers, a very sensitive but not very specific examination which, when it is positive, imposes the implementation of a spiral thoracic angioscan with injection of iodinated contrast product, or of pulmonary scintigraphy in PE, or of a Doppler in DVT. A low clinical probability, combined with a D-dimer level below the threshold of 0.5 μg/ml, makes it possible to exclude the presence of a PE or of a DVT with an excellent negative predictive value.

The use of D-dimer assaying as an exclusion test results in the need for recourse to expensive imaging tests, in order to visualize the thrombus and to diagnose the seriousness with the number and type of pulmonary arteries affected in the case of PE. In addition, such examinations prolong the patients″ stay in the emergency department, and are sources of irradiation and responsible for not insignificant cost. The angioscan may be responsible for allergic reactions to the iodinated contrast product, for renal insufficiency and for pulmonary edema.

The D-dimer antigen is a unique marker of the degradation of fibrin formed by the sequential action of thrombin, of coagulation factor FXIIIa and of plasmin as shown in FIG. 1. Specifically, thrombin cleaves fibrinogen to fibrin monomers, which polymerize and serve as a substrate for FXIIIa and for plasmin formation. Then, the thrombin remains linked to the fibrin and activates the FXIII bound to the fibrin polymers, so as to produce FXIIIa, which catalyzes the formation of covalent bonds between the D domains of polymerized fibrin. Finally, plasmin degrades polymerized fibrin so as to release fibrin degradation products (FDPs) of various molecular weights, including the end products containing the D-dimer-fragment E complex, and exposes the D-dimer antigen, which remains undetectable until it is released by plasmin. The D-dimer antigen may be present either on the FDPs originating from fibrin before it is incorporated into the fibrin clot, or high-molecular-weight complexes released after degradation of the clot by plasmin (Adam, Blood, 2009, 113(13): 2878-2887).

The D-dimer is a marker of fibrin clot formation and of fibrinolysis. Fibrinolysis plays an important role in clot stability and the thrombotic risk if it begins after clot formation. If it is initiated at the same time, there is competition between the thrombin-fibrin bond and the plasmin-fibrin bond, and therefore between fibrin formation and lysis.

The D-dimers originate from the degradation of soluble-fibrin polymers, which are markers of intravascular coagulation activation (Mirshahi. PLoS ONE, 2014, 9(3): e92379, doi:10.1371/journal.pone.0092379). In hypercoagulation states and in acute inflammatory states, the D-dimers also originate from extravascular fibrin degradation by the enzymes present locally at the inflammation sites, as shown in FIG. 2. This notion is supported by the often high levels of D-dimers in patients suffering from cancer or from acute inflammatory states (Dirix, Br. J. Cancer, 2002, 86(3): 389-395).

When fibrinolysis extends and becomes generalized (“hyperfibrinolysis”), fibrinogen degradation by plasmin generated in great excess then becomes possible. It appears in the circulation, both of the fibrin degradation products, including the D-dimers, and the fibrinogen degradation products (FgDPs), as shown in FIG. 2. Thus, high D-dimer levels are generated in cancer and in significant coagulation activation states, in combination with high FgDP levels.

The D-dimer assays currently used are not identical, since the D-dimer antigen is present on FDPs of different size and the monoclonal antibodies recognize different epitopes (Dempfle, FACT study group, Thromb. Haemost., 2001, 85(4): 671-678). The methods most commonly used are automated immunoturbidimetric methods, based on the change in turbidity of a suspension of latex microparticles coated with anti-D-dimer antigen monoclonal antibodies. In the presence of D-dimers in the sample, the latex microparticles agglutinate, indicating an increase in the turbidity of the medium, which results in an increase in absorbance by photometry at 540 nm. These plasma assays have better sensitivity than the agglutination tests in whole blood (de Groot, Thromb. Haemost., 1999, 82(6): 1588-1592), thus allowing the exclusion of VTE, but are not very specific, and cannot be used for the diagnosis of VTE.

However, no prior art method has linked the dynamic measurement of fibrin formation of a sample to D-dimers and to fibrinogen degradation products (FgDPs) generated by hypercoagulation, inflammation and hyperfibrinolysis, in order to determine the D-dimers resulting from intravascular fibrin lysis. Thus, no current method is capable of measuring the D-dimers specific for venous thrombosis in a blood sample.

The provision of an in vitro diagnosis method which is as sensitive as but more specific than the usual assaying of D-dimers could thus reduce the need for imaging examinations in patients suspected of having PE or DVT, such as a spiral thoracic angioscan with the injection of iodinated contrast product, or pulmonary scintigraphy. Such a method would make it possible to limit costs, to ensure faster treatment of patients, in particular elderly individuals and patients suffering from cancer, at high risk of thrombosis, and to allow a rapid decision about treatment to be made.

There is therefore a need to have available a method for determining D-dimers specific for venous thrombosis in a sample from a patient, said method being capable of differentiating the D-dimers of said patient that are generated by hypercoagulation, inflammation and hyperfibrinolysis, from those which result from intravascular fibrin lysis and are specific for thrombosis, as is shown in FIG. 2. Such a method must, in addition, be automated in order to be carried out in an emergency in the laboratory or able to be carried out at the patient's bedside. Furthermore, it must be reliable, reproducible and simple, and must assist in the decision about treatment.

### SUMMARY OF THE INVENTION

Surprisingly, the present inventors have developed a method capable of distinguishing the D-dimers originating from coagulation activation, from inflammation and from excess fibrinolysis, from those which are specific for thrombosis, originating from intravascular fibrin degradation, and of quantitatively measuring them. This method also allows them to be determined in an emergency situation on an automated laboratory device, a remote biology device or a portable device. The method developed is reliable, simple, quick to carry out, and reproducible. Furthermore, the inventors have refined this method such that, in its modified version, the method is applicable to all patients regardless of their age, and reduces the number of false negatives, thus making it possible to detect a venous thrombosis in the event of underlying thrombophilia, in the event of subsegmental or non-serious pulmonary embolism, in the event of myocardial infarction, in the event of cancer, in the event of infection and in the event of pregnancy, and also for elderly subjects with an underlying inflammatory susceptibility. Finally, contrary to the common assaying methods, the method according to the invention does not require any adjustment as a function of age.

The method according to the invention is based on three findings: hypercoagulation and hyperfibrinolysis are absent or reduced in thrombosis, contrary to the coagulation activation states; on the other hand, the inflammatory state is of primary importance.

More specifically, in a first aspect, the present invention relates to a first version of a method for assaying D-dimers specific for venous thromboembolism in a blood sample from a patient, said method comprising, on the one hand, the assaying of D-dimers in the sample in order to obtain the level of D-dimers in the sample (Ddi_{S}), and on the other hand, the dynamic measurement of fibrin formation in this same sample, said dynamic measurement comprising the following steps:
a) initiating the activation of coagulation in the sample without triggering it;
b) incubating the mixture obtained in step a), and triggering, in the incubated sample, the generation of thrombin and the formation of a fibrin clot;
c) measuring the time variation of at least one property of the sample obtained in b), in which the fibrin clot forms;
d) establishing the formation profile of the fibrin clot analyzed in c), and extracting from this profile the fibrin formation time (FFT) measured at the point of inflection of the tangent of the profile curve, and the value of the property (Vp_{(TA)}) of the sample measured at the time to reach (TA) the fibrin polymerization plateau;
e) calculating the level of D-dimers resulting from intravascular fibrin degradation (R):
   e1) by adjusting the level of D-dimers of the sample (Ddi_{S}) as a function of the level of D-dimers generated by hypercoagulation using FFT determined in d), in order to obtain the level of D-dimers adjusted as a function of the hypercoagulation (Ddi_{S/HC}); and
   e2) by adjusting the level of adjusted D-dimers Ddi_{S/HC} obtained in e1) as a function of the level of D-dimers generated by inflammation using Vp_{(TA)} determined in d), in order to obtain R;
f) comparing the level of D-dimers resulting from the fibrin degradation R obtained in e), with respect to a threshold, preferably to the threshold of 0.5 μg/ml;
g) determining the level of fibrinogen degradation products generated by hyperfibrinolysis (FgDP_{(HF)}) present in the coagulation activation states, using the level of D-dimers resulting from the fibrin degradation, R, obtained in e) and the level of D-dimers of the sample (Ddi_{S});
h) comparing the FgDP_{(HF)} level obtained in g) with respect to a threshold, and comparing the fibrin formation time (FFT) obtained in d) with respect to a threshold.

In some embodiments, the first version of the method according to the invention is characterized in that, in step e), calculating the level R expressed in initial fibrinogen equivalent units (FEUs) is carried out:
e1) by calculating the level of D-dimers adjusted as a function of hypercoagulation (Ddi_{S/HC}) using the following equation:

$${Ddi}_{S / HC} = {Ddi}_{s} \times \frac{FFT}{Control Time}$$

wherein the Control Time is the average fibrin clot formation time of samples from healthy patients with no suspicion of thrombosis, measured according to steps a)-d), and
e2) by calculating the level R using the following equation:

$$R = \frac{{Ddi}_{S / HC}}{{[ Fib ]}_{( Vp ( TA )}}$$

wherein [Fib]_{(Vp(TA))} is the fibrinogen concentration deduced for the value of the property Vp_{(TA)} on a standard curve having the equation:

y=a ln(x)−b,
wherein:
y is the value of the property measured at the time to reach (TA) the fibrin polymerization plateau,
x is the fibrinogen concentration,
a and b are the constants of the logarithmic equation which links the level of the fibrin plateau, and the fibrinogen concentration,
the standard curve having been established for blood samples, the fibrinogen concentration of which has been determined and the value of the property (Vp_{(TA)}) of which has been determined by steps a)-d).

In some embodiments, the first version of the method according to the invention is characterized in that, in step f), a level R obtained in e), below the threshold, preferably below the threshold 0.5 μg/ml, makes it possible to exclude a thrombosis in the patient, and wherein a level R above the threshold, preferably above the threshold of 0.5 μg/ml, is indicative of the possibility of a thrombosis in the patient.

In some embodiments, the first version of the method according to the invention is characterized in that step g) comprises steps consisting in:
g1) determining the level of FgDP corresponding to the level of D-dimers in the sample (Ddi_{S}) on a standard curve established using blood samples with known levels of FgDP and known levels of D-dimers,
g2) determine the level of FgDP corresponding to the adjusted level of D-dimers (R) obtained in step e) on the standard curve used in g1), and
g3) determining the level of FgDP generated by hyperfibrinolysis (FgDP_{(HF)}) by subtracting the level of FgDP obtained in g2) from the level of FgDP obtained in g1).

In some embodiments, the first version of the method according to the invention is characterized in that step h) comprises steps consisting in:
h1) comparing FgDP_{(HF)} to a threshold, in particular a threshold of 1 μg/ml, wherein FgDP_{(HF)} below the threshold or negative makes it possible to exclude thrombosis in the patient, and FgDP_{(HF)} above the threshold is indicative of a possibility of thrombosis in the patient,
h2) comparing FFT to a threshold, in particular to a threshold equal to [Control Time of e1)−1 standard deviation], for example a threshold of 120 seconds for a Control Time of 135 seconds, where FFT below the threshold is indicative of a patient without thrombosis but having an acute coagulation activation state, and a FFT above the threshold is indicative of a thrombosis in the patient.

In some embodiments, the first version of the method according to the invention is characterized in that:
when a thrombosis has been diagnosed in step h2), the level of D-dimers specific for venous thromboembolism (Ddi_{VTE}) is the level of D-dimers, R, obtained in step e),
when a thrombosis has been excluded in steps h1) and h2) but the level R obtained in step e) is above a threshold, preferably above the threshold of 0.5 μg/ml, the method also comprises a step consisting in calculating the level of D-dimers specific for venous thromboembolism (Ddi_{VTE}) using the following equation:

$$D d {i}_{V T E} = 0.5 \times \frac{R}{{Ddi}_{S}}$$

wherein Ddi_{S} is the level of D-dimers in the sample.

The Ddi_{VTE} level is representative of the extent of the pulmonary embolism or of the deep vein thrombosis, and therefore of the seriousness of the disease.

The present invention also relates to a second version of a method for assaying D-dimers specific for venous thromboembolism in a blood sample from a patient, said method comprising, on the one hand, the assaying of D-dimers in the sample in order to obtain the level of D-dimers in the sample (Ddi_{S}), and on the other hand, the dynamic measurement of the fibrin formation of this same sample, said dynamic measurement comprising the following steps:
a) initiating the activation of coagulation in the sample without triggering it;
b) incubating the mixture obtained in step a), and triggering, in the incubated sample, the generation of thrombin and the formation of a fibrin clot;
c) measuring the time variation of at least one property of the sample obtained in b), in which the fibrin clot forms;
d) establishing the formation profile of the fibrin clot analyzed in c), and extracting from this profile the fibrin formation time (FFT) measured at the inflection point of the tangent of the curve of the profile, and the value of the property (Vp_{(TA)}) of the sample measured at the time to reach (TA) the fibrin polymerization plateau;
e′) calculating the level of D-dimers resulting from intravascular fibrin degradation (R):
   e′1) by adjusting the level of D-dimers of the sample, Ddi_{S}, as a function of the level of D-dimers generated by inflammation using Vp_{(TA)} determined in d), in order to obtain the level of D-dimers adjusted for inflammation (Ddi_{S/I}); and
   e′2) by correcting the level of D-dimers adjusted for inflammation (Ddi_{S/I}) obtained in e′1) for the low D-dimer levels; and
classifying the sample from the patient as a function of inflammation;
f) comparing the level of D-dimers resulting from the degradation of the fibrin R obtained in e), with respect to a threshold, preferably to the threshold of 0.5 μg/ml;
g′) determining the level of D-dimers generated by hyperfibrinolysis (Ddi_{(HF)}) using the level of D-dimers in the sample (Ddi_{S}) and the level R obtained in e′) or using the level of D-dimers adjusted as a function of inflammation, Ddi_{S/I}, obtained in e′1) and the level R obtained in e′);
h′) comparing the level Ddi_{(HF)} obtained in g′) with respect to a threshold, and comparing the TA/FFT ratio with respect to a threshold, wherein TA is the time to reach the fibrin polymerization plateau obtained in d), and FFT is the fibrin formation time obtained in d).

In some embodiments, the second version of the method according to the invention is characterized in that, in step e′), the level R expressed in initial fibrinogen equivalent units (FEUs) is obtained:
e′1) by calculating the level of D-dimers adjusted as a function of inflammation (Ddi_{S/I}) by the following equation:

$$D d {i}_{S / I} = \frac{D d {i}_{S}}{{[ Fib ]}_{( Vp ( TA )}}$$

wherein [Fib]_{(Vp(TA))} is the fibrinogen concentration deduced for the value of the property Vp_{(TA)} on a standard curve having the equation:

y=a ln(x)−b,

wherein:
y is the value of the property measured at the time to reach (TA) the fibrin polymerization plateau,
x is the fibrinogen concentration,
a and b are the constants of the logarithmic equation which links the level of the fibrin plateau and the fibrinogen concentration,
the standard curve having been established for blood samples, the fibrinogen concentration of which has been determined and the value of the property Vp_{(TA)} of which has been determined by steps a)-d);
e′2) by correcting the level Ddi_{S/I} obtained in e′1) by the following equation:

R=Ddi_{S/I}+[0.5−F_{Ddi-S}]
wherein F_{Ddi-S} is a correction factor for the low D-dimer levels (<4 μg/ml), the value of which corresponds to the value of the correction factor for the level of D-dimers of the sample (Ddi_{S}) on the standard curve having the equation:

y=ax²+bx+c
wherein y is the correction factor, F,
x is the level of D-dimers,
a, b and c are the constants of the polynomial equation which links the correction factor and the level of D-dimers,
the standard curve having been established for blood samples, the level of D-dimers of which has been determined and for which the correction factor has been determined empirically so that the level Ddi_{S/I} is related back to the threshold of 0.5 μg/ml FEUs (fibrinogen equivalent units).

In some embodiments, the second version of the method according to the invention is characterized in that, in step e′), classifying the sample from the patient as a function of inflammation comprises:
calculating the ratio 1/[Fib]_{(Vp(TA))}, wherein [Fib]_{(Vp(TA))} is the fibrinogen concentration determined in e1′); and
classifying the sample from the patient in group I of patients without inflammation if the ratio 1/[Fib]_{(Vp(TA))}>0.20, or
classifying the sample from the patient in group II of patients with inflammation if the ratio 1/[Fib]_{(Vp(TA))}≤0.20.

In some embodiments, the second version of the method according to the invention is characterized in that, in step f′), a level R obtained in e′), below the threshold, preferably below the threshold of 0.5 μg/ml, makes it possible to exclude a thrombosis in the patient.

In some embodiments, the second version of the method according to the invention is characterized in that, in step g′), the level of D-dimers generated by hyperfibrinolysis (Ddi_{HF}) is calculated:
g′1) as the ratio between the level R determined in step e′) and the level of D-dimers of the sample, Ddi_{S}, using the equation:

$$D d {i}_{H F} = {( R / {Ddi}_{S} )}_{p a t i e n t} = \frac{R}{{Ddi}_{S}}$$

g′2) as the ratio between the level R determined in step e′) and the level Ddi_{S/I}, obtained in e′1), using the equation:

$$Dd {i}_{H F} = {( R / {Ddi}_{S / I} )}_{p a t i e n t} = \frac{R}{{Ddi}_{S / I}}$$

In some embodiments, the second version of the method according to the invention is characterized in that, in step h′):
the level (R/Ddi_{S})ₚₐₜᵢₑₙₜ obtained in g′1) is compared to a threshold:
h′1) by determining, for the level Ddi_{S} of the sample, the value of the ratio (R/Ddi_{S})_{standard} on a standard curve having the equation:

y=ax^{-b}
wherein x is the level of D-dimers,
y is the ratio between the level of D-dimers which result from intravascular fibrin degradation and the level of D-dimers, (R/Ddi_{S}),
a and b are the constants of the equation which links the ratio R/Ddi_{S} and the level of D-dimers,
the standard curve having been established using:
   if the sample from the patient has been classified in group I: blood samples classified in group I by step e′) and the level of D-dimers of which is known or has been determined and the level R of which has been obtained by steps a)-e′2); and
   if the sample from the patient has been classified in group II: blood samples classified in group II by step e′) and the level of D-dimers of which is known or has been determined and the level R of which has been obtained by steps a)-e′2); and
h″1) by comparing the value of the level of D-dimers generated by hyperfibrinolysis (R/Ddi_{S})ₚₐₜᵢₑₙₜ obtained in g′1) with the value of the ratio (R/Ddi_{S})_{standard} obtained in h′1), wherein:
   (R/Ddi_{S})ₚₐₜᵢₑₙₜ less than the ratio (R/Ddi_{S})_{standard} makes it possible to exclude thrombosis in the patient, and (R/Ddi_{S})ₚₐₜᵢₑₙₜ greater than or equal to the ratio (R/Ddi_{S})_{standard} is indicative of a possibility of thrombosis in the patient;
the level (R/Ddi_{S/I})ₚₐₜᵢₑₙₜ obtained in g′2) is compared to a threshold:
h′2) by determining, for the level Ddi_{S} of the sample, the value of the ratio (R/Ddi_{S/I})_{standard} on a standard curve having the equation:

y=ax^{-b}

wherein x is the level of D-dimers,
y is the ratio between the level of D-dimers which result from intravascular fibrin degradation and the level of D-dimers adjusted as a function of inflammation, (R/Ddi_{S/I}),
a and b are the constants of the equation which links the ratio R/Ddi_{S/I} and the level of D-dimers,
the standard curve having been established:
   using, if the sample from the patient has been classified in group I: blood samples classified in group I by step e′) and the level of D-dimers of which is known or has been determined, the level Ddi_{S/I} of which has been obtained by steps a)-e′1), and the level R of which has been obtained by steps a)-e′2); and
   using, if the sample from the patient has been classified in group II: blood samples classified in group II by step e′) and the level of D-dimers of which is known or has been determined, the level Ddi_{S/I} of which has been obtained by steps a)-e′1), and the level R of which has been obtained by steps a)-e′2); and
h″2) by comparing the value of the level of D-dimers generated by hyperfibrinolysis (R/Ddi_{S/I})ₚₐₜᵢₑₙₜ obtained in g′2) with the value of the ratio (R/Ddi_{S} n)_{standard} obtained in h′2), wherein:
   (R/Ddi_{S/I})ₚₐₜᵢₑₙₜ less than the ratio (R/Ddi_{S/I})_{standard} makes it possible to exclude thrombosis in the patient, and wherein (R/Ddi_{S/I})ₚₐₜᵢₑₙₜ greater than or equal to the ratio (R/Ddi_{S/I})_{standard} is indicative of a possibility of thrombosis in the patient.

In some embodiments, the second version of the method according to the invention is characterized in that, in step h′), comparing the ratio TA/FFT with respect to a threshold comprises:
h′3) calculating the ratio TA/FFT wherein TA is the time to reach the fibrin polymerization plateau determined in step d) and FFT is the fibrin clot formation time determined in step d); and
h″3) if the sample from the patient has been classified in group I: comparing the ratio TA/FFT with respect to a first threshold, in particular to a first threshold of 1.75 for a Control Time of 135 seconds, wherein:
   a ratio TA/FFT above the first threshold makes it possible to exclude thrombosis and to diagnose thrombophilia or a coagulation activation state in the patient classified in group I, and
   a ratio TA/FFT below or equal to the first threshold is indicative of a thrombosis in the patient classified in group I;
if the sample from the patient was classified in group II: comparing the ratio TA/FFT with respect to a second threshold, in particular to a second threshold of 1.65 for a Control Time of 135 seconds, wherein:
   a ratio TA/FFT above the second threshold makes it possible to exclude thrombosis and to diagnose thrombophilia or a coagulation activation state in the patient classified in group II, and
   a ratio TA/FFT below or equal to the second threshold is indicative of a thrombosis in the patient classified in group II,
      wherein the Control Time is the average time for fibrin clot formation of samples from healthy subjects who do not present a suspicion of thrombosis, measured according to steps a)-d).

In some embodiments, the second version of the method according to the invention is characterized in that:
when a thrombosis has been diagnosed in step h″3), the level of D-dimers specific for venous thromboembolism (Ddi_{VTE}) is the level of D-dimers, R, obtained in step e′),
when a thrombosis has been excluded in steps h″1), h″2) and h″3), but the level R obtained in step e′) is above the threshold, preferably above the threshold of 0.5 μg/ml, the method also comprises a step consisting in calculating the level of D-dimers specific for venous thromboembolism (Ddi_{VTE}) using the following equation:

$$D d {i}_{V T E} = 0.5 \times \frac{R}{D d {i}_{S}}$$

wherein Ddi_{S} is the level of D-dimers in the sample.
   The level Ddi_{VTE} is representative of the extent of the pulmonary embolism or of the deep vein thrombosis, and therefore of the seriousness of the disease.

In some embodiments, the first or the second version of the method according to the invention is characterized in that the blood sample has a volume of between 1 μl and 300 μl, preferably between 50 μl and 200 μl. Preferably, the blood sample is undiluted.

The assaying of the D-dimers of the sample can be carried out according to an immunoturbidimetric or immunoenzymatic method.

In some embodiments, step a) of an assaying method according to the invention is carried out by mixing the blood sample from the patient with tissue factor and optionally phospholipids, preferably by mixing the blood sample from the patient with tissue factor and phospholipids. The tissue factor of step a) can be present in a concentration of between 0.5 and 5 pM, preferably 2 pM. The mixture of step a) can comprise calcium ions in order to trigger thrombin generation and fibrin formation.

In some embodiments, step b) of an assaying method according to the invention is characterized in that it comprises incubating the mixture obtained in step a) for a time of between 20 seconds and 400 seconds, preferably between 60 seconds and 300 seconds, at a temperature between 30° C. and 40° C.

In some embodiments, in step b), triggering thrombin generation and fibrin clot formation is carried out by adding calcium ions to the sample incubated.

In some embodiments, the blood sample used in an assaying method according to the invention is a plasma sample. The plasma sample can be a platelet-poor plasma sample. In these embodiments, in step c), measuring the time variation of at least one property of the sample obtained in b) is carried out by measuring the time variation of the optical density (DOD) at a wavelength of between 350 and 800 nm, preferably at the wavelength of 540 nm. Preferably, the measurement of the optical density of step c) is carried out at the same wavelength as that used for assaying the D-dimers, 540 nm.

In other embodiments, the blood sample used in an assaying method according to the invention is a whole-blood sample. The whole-blood sample can be a citrated whole-blood sample. In these embodiments, in step c), measuring the time variation of at least one property of the sample obtained in b) is carried out by thromboelastography, by rheometry or by image analysis.

In some embodiments, an assaying method according to the invention is characterized in that at least steps c) and d) are carried out on an automated diagnostic device or on a remote biology analyzer, preferably on a coagulation analyzer.

In a second aspect, the present invention relates to an in vitro method for diagnosing venous thromboembolism (VTE) in a patient, comprising steps consisting in:
carrying out an assay of D-dimers specific for VTE in a blood sample from the patient using a method for assaying D-dimers specific for VTE described herein; and
providing a diagnosis regarding the patient.

In one in vitro diagnosis method according to the invention, the diagnosis regarding the patient is (i) exclusion of thrombosis, (ii) acute coagulation activation state, or (iii) thrombosis.

In the embodiments wherein the patient is an elderly individual, a patient suffering from cancer, a patient suffering from an infection or a patient suffering from thrombophilia, the assaying of the D-dimers specific for venous thromboembolism is carried out using the second version of the assaying method of the invention. In this case, the diagnosis regarding the patient is (i) exclusion of thrombosis, (ii) acute coagulation activation state, (iii) thrombophilia, or (iv) thrombosis.

A more detailed description of some preferred embodiments of the invention is given below.

### DETAILED DESCRIPTION OF THE INVENTION

As mentioned above, the present invention relates to a method for determining the level of D-dimers specific for thrombosis in a biological sample from a patient and the application thereof in the diagnosis of venous thromboembolism (pulmonary embolism or deep vein thrombosis) or the identification of a coagulation activation state.

### I—Method for Assaying D-Dimers Specific for Venous Thromboembolism

An assaying method according to the invention comprises two main steps: on the one hand, assaying of the D-dimers, and on the other hand, the dynamic measurement of fibrin formation. These main steps are carried out on a blood sample from the patient.

A. Blood Sample from the Patient

The term “patient” as used herein, denotes a human being. The term “patient” does not denote a particular age, and therefore encompasses children, adolescents and adults, including elderly individuals. Generally, the patient is a subject who is suspected of having a pulmonary embolism or deep vein thrombosis, for instance a patient treated in the emergency department in hospital for dyspnea and/or chest pain. Such a patient may, moreover, have no known medical condition. Alternatively, such a patient may be known to have a predisposition to thrombosis (for example, a patient suffering from cancer, a pregnant woman or a woman who has just given birth, a patient in the post-operative phase, a subject who is traveling or who has just taken a trip, in particular a long trip); a patient presenting a hypercoagulation state (such as a patient suffering from thrombophilia, a patient suffering from renal insufficiency, a patient having undergone a trauma, a fall or a fracture, or an elderly subject); a patient presenting a coagulation activation state (such as a patient suffering from infection or from sepsis, a patient suffering from pneumopathy, from bronchitis or from respiratory insufficiency, a patient suffering from inflammatory disease, a patient suffering from gastritis, a patient suffering from cardiomyopathy, or a patient suffering from a history of stroke). In the context of the present invention, the term “normal patient” or “healthy patient” is used when the patient has no suspicion of thrombosis.

The method according to the invention uses a simple blood biological sample from the patient. A blood sample is taken from the patient's vein for the purpose of harvesting the blood sample. Preferably, this blood biological sample is used undiluted in the method.

In some embodiments, the method according to the invention is carried out on a sample of whole blood (that is to say of blood with all its constituents). The whole blood may be citrated. In this case, the whole blood taken is harvested in a citrated tube.

In other embodiments, the method according to the invention is carried out on a plasma sample obtained from the blood sample. The methods for obtaining plasma from human blood are known in the art. Preferably, the biological sample is a platelet-poor plasma (PPP) sample. In this case, it can in particular be obtained by centrifugation of the citrated tube, comprising the patient's blood sample, for 15 minutes, at a speed of from 2000 to 2500 g, in a thermostated centrifuge at a temperature of between 18 and 22° C.

If the platelet-poor plasma sample must be stored, it is possible to use the following protocol, which consists in:
rapidly decanting the plasma, leaving approximately 0.5 cm of plasma above the cell layer of white blood cells and platelets;
recovering the plasma in a hemolysis tube or a plastic tube;
centrifuging this tube for 15 minutes, at a speed of from 2000 to 2500 g, in a thermostated centrifuge at a temperature of between 18 and 22° C.;
aliquoting in fractions of from 0.5 to 1 ml without taking the bottom of the tube (which contains the cell debris); then
rapidly freezing at a temperature of between −70° C. and −90° C., preferably at −80° C.

The assaying according to the invention can be carried out on any appropriate volume of blood sample. Generally, in the present invention, a small volume of blood sample is used. For example, the blood biological sample has a volume of between 1 μl and 300 μl, preferably between 50 μl and 200 μl, preferably a volume of approximately 200 μl for a final volume of 300 μl after addition of the reagents (see below), preferably a volume of approximately 100 μl for a final volume of 150 μl after addition of the reagents. Such a volume is in fact sufficient for the analysis on a routine instrument, but may be reduced on a remote biology device, provided that the sample volume to final volume ratio (i.e. ratio of approximately 2:3) is adhered to. It may be further reduced to a volume of between 5 μl and 20 μl, preferably 10 μl, in the case of a portable device of which the reagent is freeze-dried and reconstituted by the sample at the time of use at the bedside of the patient, provided that the same final reaction concentrations are obtained.

### B. Assaying of the D-Dimers of the Sample

In a method according to the invention, the first main step (the assaying of the D-dimers in the blood sample) can be carried out by any appropriate method. In some embodiments, a usual assaying of the D-dimers of the sample will be carried out. The expression “usual assaying of the D-dimers of the sample” is intended to mean assaying carried out according to an immunoturbidimetric method, such as the latex method, or an immunoenzymatic method, such as the ELISA (“enzyme-linked immunosorbent assay”) method or the ELFA (“enzyme-linked fluorescent assay”) method. The immunoturbidimetric methods are based on the change in turbidity of a solution comprising the D-dimers. Typically, an immunoturbidimetric method comprises (i) mixing the blood sample from the patient with a suspension of latex microparticles coated with anti-D-dimer antigen monoclonal antibodies (“latex method”); then (ii) monitoring the turbidity of the mixture, in particular at a given wavelength, typically at 540 nm. An immunoenzymatic method comprises capturing the D-dimers of the invention with anti-D-dimer antigen monoclonal antibodies and then revealing them with labeled secondary antibodies.

### C. Dynamic Measurement of Fibrin Formation

The method according to the invention is based on three findings: hypercoagulation and hyperfibrinolysis are absent or reduced in thrombosis, contrary to coagulation activation states; on the other hand, the inflammatory state is of primary importance.

As indicated above, the present inventors have developed two versions of the method for assaying D-dimers specific for venous thromboembolism. The dynamic measurement of fibrin formation according to the first version of the method of the invention contains several steps: steps a), b), c), d), e), f) and g), and a diagnostic step, step h). The dynamic measurement of fibrin formation according to the second version of the method according to the invention contains the same steps a), b), c) and d) as the first version, and steps e′), f) and g′), and a diagnostic step, step h′), which differ from those of the first version. These steps are described in detail below.

### Steps of the Method

Step a)

Step a) of the assaying method according to the invention consists in initiating the activation of coagulation in the blood sample without triggering it. The initiation of coagulation activation can be carried out by any appropriate method known in the art, whether via the intrinsic pathway which consists of the activation of Hageman factor or coagulation factor XII (which is what occurs when said factor comes into contact with collagen stripped from a lesioned vessel), or via the extrinsic pathway, which is initiated by tissue factors during a tissue lesion and results in blood coagulation. For example, via the extrinsic pathway, step a) can comprise mixing the blood biological sample with tissue factor and optionally phospholipids. In some preferred embodiments, step a) comprises mixing the blood sample with tissue factor and optionally phospholipids in order to initiate the activation of the intrinsic pathway of coagulation without triggering it. In this case, the blood biological sample is mixed with a solution of tissue factor (TF), preferably human tissue factor, and of phospholipids (PLs), which have preferably been semi-purified or purified. The tissue factor and the phospholipids are preferably freeze-dried so as to be reconstituted by the blood biological sample, in particular in the case of a remote biology device or of a portable device.

The mixture of step a) can comprise a final concentration of phospholipids of from 2 to 5 μM in the mixture, preferably a concentration of approximately 4 μM. Preferably, the tissue factor is used in an amount such that its final concentration in the mixture with the blood biological sample is between 0.5 and 20 pM, preferably between 1 and 5 pM, for example 2 pM.

The mixture of step a) may also comprise calcium ions. The calcium ions may be present at a final concentration of from 15 to 20 mM, preferably of 17 mM.

In some embodiments, step a) of the method according to the invention comprises:

a1) introducing tissue factor into a solution of phospholipids and optionally of calcium ions, then

a2) mixing the solution obtained in a1) with the blood biological sample.

In other embodiments, step a) of the method according to the invention comprises:

a1) introducing phospholipids into a solution of tissue factor, and optionally adding calcium ions, then

a2) freeze-drying the solution obtained in a1) so as to be reconstituted with the blood biological sample.

At the end of step a), a mixture of at least tissue factor with the biological sample of plasma or a mixture of at least the tissue factor with the biological sample of whole blood is obtained for determining the fibrin formation profile up until the polymerization plateau.

Step b)

Step b) consists in incubating the mixture obtained in step a) then in triggering, in the incubated sample, thrombin generation and fibrin clot formation. The triggering of thrombin generation and of fibrin clot formation can be carried out by any method known in the art. This triggering involves a complex cascade of coagulation factors of the intrinsic or intrinsic pathway, which results in the conversion of fibrinogen to polymerized fibrin, thereby creating the fibrin clot. In some particular embodiments, this triggering is carried out by adding calcium ions to the mixture obtained.

Step b) thus comprises incubating the mixture obtained in step a). The incubation can be carried out under any appropriate time and temperature conditions. This incubation can typically be carried out for a time of between 20 seconds and 400 seconds, preferably between 60 seconds and 300 seconds, preferably of 300 seconds on the routine instrument, at a temperature of between 30° C. and 40° C., preferably at a temperature of approximately 37° C. This incubation time can be shortened to less than 60 seconds, preferably less than 30 seconds with reduced volumes, in particular with the use of a remote biology instrument, preferably with the use of a portable device.

Calcium ions are then added to the incubated mixture. These calcium ions can be added in the form of a CaCl₂ solution, at a concentration of approximately 0.1 M.

Thrombin generation and fibrin clot formation are triggered through the addition of calcium, in the presence of tissue factor.

Step c)

Step c) consists in measuring the time variation of at least one property of the sample in which the fibrin clot forms. The property may be any optical or physical property which makes it possible to monitor fibrin clot formation.

In some embodiments, in particular in cases where the biological sample used is a plasma sample, the property measured is preferably an optical property, in particular the optical density (OD) (also called absorbance) at at least one wavelength. The wavelength may be between 350 nm and 800 nm. Preferably, the optical density measurement is carried out at the wavelength of 540 nm, which is the one used for the usual assaying of the D-dimers.

Thus, for example, during step c), the fibrin clot formation is monitored dynamically, for example every 2 seconds or less, by measuring the optical density at at least one wavelength of between 350 nm and 800 nm, preferably at 540 nm, and for a period of between 1 and 10 minutes, preferably 10 minutes. During this period, the fibrin clot formation is therefore analyzed, in particular by the variations in optical density (DOD) compared to the optical density at the base time (or initial time tᵢ), which may, for example, be between 10 and 20 seconds. Thus, at the analysis wavelength λ, and at the measurement time t, DOD(λ)ₜ corresponds to the optical density value measured at time t at the wavelength λ. (OD(λ)ₜ) minus the optical density value measured at time tᵢ, at the wavelength λ (OD(λ)ₜᵢ) and calculated as follows:

DOD(λ)ₜ=OD(λ)ₜ−OD(λ)ₜᵢ

Alternatively, the property of the sample in which the fibrin clot forms that is dynamically measured during step c) of the method according to the invention is a physical property, which may be the turbidity, the elasticity, the viscosity, the viscoelasticity, the rigidity modulus, etc.

Thus, in other embodiments, in particular in cases where the biological sample used is a whole-blood sample, the physical property of the fibrin clot that is dynamically monitored during step c) is preferably measured by thromboelastography, by rheometry or by image analysis. In general, the fibrin clot formation is monitored by the time variation of the physical properties of the clot, until the amplitude at the time to reach the fibrin polymerization plateau has been reached, for example for a period of less than or equal to 10 minutes, preferably less than 5 minutes.

Step d)

Step d) of the method according to the invention consists first of all in establishing the formation profile of the fibrin clot analyzed during step c).

The expression “formation profile of the fibrin clot” is intended to mean the change in at least one optical or physical property of the clot as a function of time during fibrin formation up until the fibrin polymerization plateau.

The dynamic measurement of fibrin formation, followed by the determination, as a function of time, of at least one property of a blood sample in which thrombin generation and fibrin clot formation is initiated, provides three pieces of data:
the fibrin clot formation time (FFT),
the time to reach (TA) the fibrin polymerization plateau, and
the value of the property measured at time TA, (Vp_{(TA)}).

Thus, step d) therefore consists in extracting from the fibrin clot formation profile established, the fibrin formation time (FFT) measured at the point of inflection of the tangent to the curve and the value of the optical or physical property (Vp_{(TA)}) measured at the time to reach (TA) the fibrin polymerization plateau. Those skilled in the art will understand that, alternatively, the value of the optical or physical property can be measured at the time to reach more than 90% of the polymerization plateau, preferably at the time to reach more than 95% of the polymerization plateau, for example 96%, or 97%, or 98% or else 99%.

In the case where the property measured is an optical density, that is to say in particular in the case of a plasma sample, the fibrin formation time (FFT) measured at the inflection point of the tangent to the curve, and the DOD_{(TA)} of the sample measured at the time to reach (TA) the fibrin polymerization plateau, are extracted from the fibrin clot formation profile (sigmoid profile, which resembles an S-shaped curve with a plateau).

In the case where the physical property is measured by thromboelastography, that is to say in particular the case of a whole-blood sample, the fibrin clot formation time (FFT) measured at the inflection point of the tangent to the curve, and the amplitude (A_{(TA)}) at the time to reach (TA) the fibrin polymerization plateau, are extracted from the fibrin clot formation profile (profile which has the shape of a tuning fork).

In the case where the physical property is measured by rheometry or by image analysis, that is to say in particular in the case of a whole-blood sample, the fibrin formation time (FFT) measured at the inflection point of the tangent to the curve, and the amplitude (A_{(TA)}) at the time to reach (TA) the fibrin polymerization plateau, are extracted from the fibrin clot formation profile (sigmoid profile, which resembles an S-shaped curve with a plateau).

This is in particular illustrated:
in FIG. 5a, with a plasma from a normal (healthy) patient and a plasma from a patient with hypercoagulation, and
in FIG. 5b, with a whole blood from a normal patient and a whole blood from a patient with hyperfibrinogenemia.

The fibrin formation time (FFT) determined at the inflection point of the tangent to the curve and also the time to reach (TA) the start of the fibrin plateau are shortened proportionally to the hypercoagulation, whether with hypercoagulant plasma for the method using the optical density measurement, or with whole blood having a high fibrinogen level, for the method where the physical property is measured by thromboelastography, by rheometry or by image analysis.

The level of the fibrin polymerization plateau, expressed either by the DOD_{(TA)} at the time to reach the plateau, or the amplitude (A_{(TA)}) at the time to reach the plateau, increases with inflammation, represented either by the level of fibrinogen in FIG. 5, or by the level of C-reactive protein (CRP) in FIG. 6(A) and the level of fibrinogen in FIG. 6(B), in the samples from patients for whom the two proteins (CRP and fibrinogen) have been assayed.

The value of OD at the time to reach the plateau is lower for the plasma from a patient with hypercoagulation than for the plasma from a normal patient (3.5 g/1), since it contains less fibrinogen (3.0 g/1).

The amplitude A at the time to reach the plateau is higher for whole blood having a high level of fibrinogen (5 g/l) than for normal blood which has a normal level of fibrinogen (2.5 g/l).

The time to reach the fibrin polymerization plateau is proportional to the fibrin formation time, for all the patients with a suspicion of thrombosis, as shown in FIG. 7(A) for the patients without active cancer, whether or not they have a venous thrombosis, and in FIG. 7(B) for the patients with an active cancer. This time to reach the plateau does not exceed 8 minutes, regardless of the patients, in the absence of anticoagulant treatment.

### First Version of the Method Steps e), f), g) and h)

The first method according to the invention makes it possible to determine the level of D-dimers specific for venous thromboembolism (VTE), that is to say the level of D-dimers originating specifically from intravascular fibrin degradation, by adjusting the level of D-dimers of the sample as a function:
of the level of D-dimers generated by hypercoagulation, after initiation of thrombin and fibrin generation;
of the level of D-dimers generated by inflammation; and
of the level of D-dimers generated by hyperfibrinolysis.

Such a method makes it possible to determine the level of D-dimers specific for venous thromboembolism for the patient in question, this being in a very short time (i.e. less than 10 minutes) as described in particular in example 1. Specifically, the various plasmas of patients with and without pulmonary embolism, with and without deep vein thrombosis, and with or without a coagulation activation state, are discriminated in less than 10 minutes, on their fibrin clot formation profile after initiation of thrombin generation.

Step e)

The present inventors have first of all developed a first version of the method in which step e) consists in calculating the level of D-dimers which result from intravascular fibrin degradation by adjusting the level of D-dimers of the sample as a function of the level of D-dimers generated by hypercoagulation and as a function of the level of D-dimers generated by inflammation. More specifically, the level of D-dimers resulting from intravascular fibrin degradation (R) is calculated:

e1) by adjusting the level of D-dimers of the sample (Ddi_{S}) as a function of the level of D-dimers generated by hypercoagulation using FFT determined in d), in order to obtain the level of D-dimers adjusted as a function of hypercoagulation, and

e2) by adjusting the level of D-dimers adjusted as a function of hypercoagulation calculated in e1) as a function of the level of D-dimers generated by inflammation using Vp_{(TA)} determined in d), in order to obtain R.

Specifically, the present inventors have observed that there is a systematic lengthening of the fibrin formation time (FFT) in all the samples from patients with pulmonary embolism (PE) and/or deep vein thrombosis (DVT), compared with the samples from patients without thrombosis, as shown in example 1. Advantage is taken of the linear example of example 2 in order to adjust the D-dimers as a function of the D-dimers generated by hypercoagulation.

The level of D-dimers adjusted as a function of hypercoagulation (Ddi_{S/HC}) is determined from the level of D-dimers of the sample (Ddi_{S}) by the following formula:

$${Ddi}_{S / HC} = {Ddi}_{S} \times \frac{FFT}{Control Time}$$

wherein the Control Time is the average time of fibrin clot formation of samples from normal healthy subjects, who do not have a suspicion of thrombosis, measured by steps a)-d) of the method according to the invention.
This is shown in particular in example 3. Thus, step e) comprises a step of calculating the D-dimers, from the fibrin formation time (FFT) measured in d) so as to take into account the D-dimers resulting from hypercoagulation.

The level of D-dimers which result from intravascular fibrin degradation (R) is calculated by adjusting the level of D-dimers adjusted as a function of hypercoagulation (Ddi_{S/HC}), as a function of inflammation, by the following formula:

$$R = \frac{{Ddi}_{S / HC}}{{[ Fib ]}_{( Vp ( TA ) )}}$$

wherein [Fib]_{(Vp(TA))} is the fibrinogen concentration deduced for the value of the property (Vp_{(TA)}) on the standard curve having the equation:

y=a ln(x)−b,

wherein:
y is the value of the property measured at the time to reach (TA) the fibrin polymerization plateau,
x is the fibrinogen concentration,
a and b are the constants of the logarithmic equation which links the level (the value or the amplitude) of the fibrin plateau, and the fibrinogen concentration,
the standard curve having been established using blood samples, the fibrinogen concentration of which has been determined and the value of the property (Vp_{(TA)}) of which has been determined by steps a)-d) of the method according to the invention.

The level of D-dimers which result from intravascular fibrin degradation (R) is thus expressed in initial fibrinogen equivalent units (FEUs).

As indicated above, in the case of a method based on the measurement of optical density, for example in the case of a plasma sample, Vp_{(TA)} is DOD(T_{A}), the optical density measured at the time to reach (TA) the fibrin polymerization plateau, and in the case of the method on the basis of where the physical property is measured by thromboelastography, by rheometry or by image analysis, for example in the case of a whole-blood sample, Vp_{(TA)} is A_{(TA)}, the amplitude measured at the time to reach (TA) the fibrin polymerization plateau.

Examples of standard curves allowing the determination of the level of inflammation (that is to say the fibrinogen concentration) are presented in FIGS. 9(A)-(B) and example 4 in the case of a method based on the measurement of DOD, and in FIG. 9(C) and example 4 in the case of a method based on a measurement of physical property by thromboelastography, respectively.

The level of D-dimers obtained is thus expressed in initial fibrinogen equivalent units (FEUs).

Step f)

Step f) of the first version of the method according to the invention consists in comparing the level of D-dimers resulting from fibrin degradation obtained in e), compared to a threshold, in order to determine the probability of a venous thrombosis, in particular of a pulmonary embolism (PE) or of a deep vein thrombosis (DVT), in the sample from the patient. Preferably, the threshold is 0.50 μg/ml. Specifically, preferably, since the amount of D-dimers generated by plasmin is approximately 50% of the FEUs (fibrinogen equivalent units) with the 8D2 and 2.1.16 antibodies used, the positivity threshold of the measurement is thus 0.50 μg/ml. Preferably, this threshold is the same as that of the usual assay of D-dimers.

A level of D-dimers resulting from fibrin degradation, R, obtained in e) which is below the threshold (preferably the threshold of 0.5 μg/ml) makes it possible to exclude a thrombosis in the patient. A level of D-dimers resulting from fibrin degradation, R, obtained in e) which is above the threshold (preferably the threshold of 0.5 μg/ml) is indicative of the possibility of a thrombosis in the patient.

In particular, among the patients with a level of D-dimers obtained in e) determined by the ratio R and expressed in FEUs (fibrinogen equivalent units), more than 90% of the patients without thrombosis have a negative level (i.e. below the threshold of 0.5 μg/ml) and all the patients without thrombosis have a positive level (that is to say above the threshold of 0.5 μg/ml) ranging from 0.50 μg/ml to 10.5 μg/ml, as shown in table 4. Among the patients without thrombosis, found to be falsely positive (<10%) with a level of 0.50 μg/ml to 2.7 μg/ml, one third have a level <0.60 μg/ml, one third have a cancer, and one third have a coagulation activation state (fracture, thrombotic microangiopathy, pregnancy or post-operative). Thus, 80% of the patients with a falsely positive level of D-dimers are rendered negative with the adjustment of the D-dimers as a function of D-dimers generated by hypercoagulation and inflammation.

Generally, when the comparison carried out in step 0 makes it possible to exclude thrombosis in the patient, the method can be stopped at step f). If the opposite is true, the method is continued.

Step g)

The first version of the method according to the invention then comprises a step g) of determining the level of fibrinogen degradation products (FgDPs) generated by the hyperfibrinolysis present in the coagulation activation states. Thus, step g) consists in:

g1) determining the level of FgDPs from the level of D-dimers in the sample (Ddi_{S}),

g2) determining the level of FgDPs from the adjusted level of D-dimers (R) obtained in step e), and

g3) calculating the level of FgDPs generated by hyperfibrinolysis (FgDP_{HF}) by subtracting the level of FgDPs obtained in g2) from the level of FgDPs obtained in g1).

Specifically, high levels of D-dimers are generated in cases of cancer and in significant coagulation activation states, in combination with high levels of fibrinogen degradation products (FgDPs), formed under the action of plasmin present in a large amount.

In particular, the level of D-dimers of the sample determined by usual assaying and also the level of D-dimers adjusted as a function of the D-dimers generated by hypercoagulation and inflammation according to the method of the invention carried out on plasma samples (step e) above) correlate perfectly with the level of FgDPs measured by the latex method in these samples, and resulting from hyperfibrinolysis, as shown in FIG. 10; this being whatever the D-dimer reagent used, as shown in FIG. 11. The same is true for the level of D-dimers adjusted as a function of the D-dimers generated by hypercoagulation and inflammation according to the method of the invention carried out on whole-blood samples. The level of FgDPs can thus be calculated by linear regression from the level of D-dimers obtained.

In significant coagulation activation states, the inventors have observed that:
hyperfibrinolysis is considerable and generates large amounts of FgDPs,
the FgDPs generated from fibrinogen are systematically lower in the samples originating from patients with pulmonary embolism (PE) and/or deep vein thrombosis (DVT) than in the samples originating from patients without thrombosis. Thus, a positive difference between the FgDPs generated as a function of the usual D-dimers and those generated as a function of the adjusted D-dimers obtained in e) makes it possible to distinguish the patients with thrombosis from those without thrombosis, at a threshold which is 1 μg/ml in example 6; this being whatever the D-dimer reagent used, as shown in FIG. 11,
the fibrin formation time (FFT) is shortened in coagulation activation states, as described in example 1, at the threshold of the Control Time−1 standard deviation. Advantage is taken of this to exclude thrombosis and to turn attention to an acute coagulation activation state, in the presence of significant hyperfibrinolysis, as described in example 6.

This makes it possible to directly and rapidly determine the probability of a pulmonary embolism or of a deep vein thrombosis and/or to turn attention to a coagulation activation in said sample from the patient, as shown in examples 5, 6 and 7. More preferentially, this makes it possible, in the presence of high levels of D-dimers, to increase the probability of PE and of DVT, or even to turn attention to the extent of the pulmonary embolism or of the venous thrombosis as shown in example 8.

Thus, a positive difference between the FgDPs generated as a function of the level of D-dimers of the sample (Ddi_{S}) and those generated as a function of the level of adjusted D-dimers (R) obtained in e) makes it possible to distinguish the patients with a possibility of thrombosis from those without thrombosis, at a predetermined threshold. In the case of the examples presented below, the inventors have predetermined a threshold of 1 μg/ml.

More specifically, step g) consists in:

g1) determining the level of FgDPs corresponding to the level of D-dimers in the sample (Ddi_{S}) on a standard curve established using blood samples, with known levels of FgDPs and known levels of D-dimers,

g2) determining the level of FgDPs corresponding to the adjusted level of D-dimers (R) obtained in step e) on this same standard curve, and

g3) calculating the level of FgDPs generated by hyperfibrinolysis (FgDP_{(HF)}) by subtracting the level of FgDPs obtained in g2) from the level of FgDPs obtained in g1).

Step h)

Finally, step h) of the first version of the method according to the invention comprises comparing the level of FgDPs generated by hyperfibrinolysis (FgDP_{(HF)}) obtained in g) with respect to a threshold and comparing the fibrin formation time (FFT) obtained in d) with respect to a threshold.

More specifically, step h) comprises a first step consisting in:

h1) comparing FgDP_{(HF)} to a threshold (for example a threshold of 1 μg/ml), wherein FgDP_{(HF)} below the threshold or negative makes it possible to exclude thrombosis in the patient, and wherein FgDP_{(HF)} above the threshold is indicative of a possibility of thrombosis in the patient.

Those skilled in the art will recognize that the threshold is a predetermined threshold which is established using a large number of samples. On the basis of the samples studied, the inventors have used a predetermined threshold of 1 μg/ml.

Step h) then comprises a second step consisting in:

h2) comparing FFT to a threshold, in particular a threshold equal to [Control Time−1 standard deviation], for example a threshold of 120 seconds for a Control Time of 135 seconds, wherein FFT below the threshold is indicative of a patient without thrombosis but having an acute coagulation activation state, and FFT above the threshold is indicative of a thrombosis in the patient, and wherein the Control Time is defined in step e1).

Here again, those skilled in the art will recognize that the threshold is a predetermined threshold which is established using a large number of samples. On the basis of the samples studied, the inventors have used a predetermined threshold of 120 seconds for a Control Time of 135 seconds.

Thus, 92% of the patients with a falsely positive level of D-dimers, that is to say 97% of the patients without thrombosis, are rendered negative with the adjustment of the D-dimers according to step h), in particular as shown in FIG. 12, whatever the reagent for assaying the D-dimers as shown in FIG. 13.

Preferably, high levels of D-dimers specific for venous thrombosis, obtained at the end of the method, are representative of the extent of the pulmonary embolism (PE) or of the deep vein thrombosis (DVT). Thus, in the case where a thrombosis is diagnosed in the patient by means of a method according to the invention, the level of D-dimers, R, obtained in step e), related back to a clinically used scale, that is to say with respect to the threshold of 0.5 μg/ml, is proportional to the extent of the pulmonary embolism or of the venous thrombosis, and therefore to the seriousness of the disease. Indeed, the diagnosis of seriousness is usually determined in imaging by the number and type of pulmonary arteries affected.

More specifically, in the case where a thrombosis is diagnosed in the patient in step h2) of a method according to the invention, the level of D-dimers specific for venous thromboembolism (Ddi_{VTE}) is the level of D-dimers, R, obtained in step e).

In the case where a thrombosis is excluded in the patient in steps h1) and h2) of a method according to the invention, but the level R obtained in step e) is above the threshold, preferably above the threshold of 0.5 μg/ml, the level of D-dimers specific for venous thromboembolism (Ddi_{VTE}) is calculated by:

$${Ddi}_{VTE} = 0.5 \times \frac{R}{{Ddi}_{S}}$$

wherein R is the level of D-dimers obtained in step e) and Ddi_{S} is the level of D-dimers of the sample. This calculation makes it possible to relate the level of D-dimers specific for venous thromboembolism back to a level below the clinically used threshold.

The levels of D-dimers specific for venous thrombosis thus provided at the end of the method are then representative of the extent of the pulmonary embolism or of the deep vein thrombosis.

The expression “level representative of the extent of the PE or of the location of the DVT” is intended to mean that the level of D-dimers specific for venous thrombosis correlates directly with the extent of the PE (i.e. with its seriousness) or with the location of the DVT. The term “high levels” is intended to mean levels of approximately 1 μg/ml to 4 μg/ml or more, as shown in table 7.

Second Version of the Method Steps e′), f′), g′) and h′)

The second version of the method according to the invention which makes it possible to determine the level of D-dimers specific for venous thromboembolism (VTE) comprises steps a), b), c) and d) identical to those of the first method and steps e′), f′), g′) and h′) different than those of the first version.

Step e′)

A second variant of step e) has been developed by the inventors, who noted that, in the majority (75%) of cases of venous thromboembolism (VTE), the levels of D-dimers are less than 4 μg/ml. Hypercoagulation and hyperfibrinolysis are absent or reduced in thrombosis, contrary to coagulation activation states. On the other hand, the inflammatory state is of primary significance. Specifically, for 80% of VTEs, half are in a first group of patients without inflammation (fibrinogen concentration: 3.0-4.5 g/1) and half are in a second group of patients with subnormal inflammation (fibrinogen concentration: 4.5-6.0 g/1). Furthermore, the time to reach (TA) the fibrin clot polymerization plateau is faster in thrombosis, and makes it possible to differentiate a VTE from a coagulation activation state or from a thrombophilia.

Consequently, step e′) of the second version of the method according to the invention consists in:
calculation the level of D-dimers which result from intravascular fibrin degradation:
   e′1) by adjusting the level of D-dimers of the sample as a function of the level of D-dimers generated by inflammation, and
   e′2) by correcting this adjusted level for low Ddi levels (<4 μg/ml), and
classifying the blood sample from the patient as a function of the inflammation.

Thus, compared with step e), in step e′), the adjustment of the level of D-dimers of the sample as a function of the level of D-dimers generated by hypercoagulation using the fibrin formation time (FFT) is deleted. This makes it possible to detect a thrombosis in the case of underlying thrombophilia for which the FFT is short (FFT<[Mean Control Time−1 standard deviation], for example FFT≤120 seconds for a control at 135 seconds) and the prevalence is 2.5 per 1000.

The correction of the adjusted level for the low Ddi levels makes it possible to detect a thrombosis:

(i) in the event of subsegmental or non-serious pulmonary embolism, in which the D-dimers are generally low (≤1.5 μg/ml) and the prevalence is in the region of 20% of PEs, and

(ii) in the event of pulmonary infarction (pulmonary embolism complications), in which the inflammation is significant (≥5 g/1) and the prevalence is in the region of 15% of PEs.

Finally, the classification of the sample from the patient as a function of inflammation makes it possible to determine a thrombosis in the case of cancer (20% of patients), of infection (10% of patients), and of elderly individuals with an underlying inflammatory susceptibility.

More specifically, in step e′), the level of D-dimers which result from intravascular fibrin degradation R is determined:

e′1) by calculating Ddi_{S/I}, the level of D-dimers adjusted as a function of inflammation, from the level of D-dimers of the sample (Ddi_{S}) using the value of the property measured at the time to reach the polymerization plateau, Vp_{(TA)}, determined in step d); and

e′2) by correcting the level Ddi_{S/I} obtained in e′1) for the levels of D-dimers<4 μg/ml, in order to determine the level of D-dimers which result from intravascular fibrin degradation (R).

Step e′1). Thus, step e′1) consists in calculating the level of D-dimers adjusted as a function of inflammation, Ddi_{S/I}, from the level of D-dimers of the sample (Ddi_{S}) using the value of the property measured at the time to reach the polymerization plateau, Vp_{(TA)}, determined in step d), by the following equation:

$${Ddi}_{S / I} = \frac{{Ddi}_{S}}{{[ Fib ]}_{( Vp ( TA ) )}}$$

wherein [Fib]_{(Vp(TA))} is the fibrinogen concentration deduced for the value of the property (Vp_{(TA)}) on the standard curve having the equation y=a ln(x)−b, wherein:
y is the value of the property measured at the time to reach (TA) the fibrin polymerization plateau,
x is the fibrinogen concentration,
a and b are constants for the logarithmic equation which links the level (the value or the amplitude) of the fibrin plateau and the fibrinogen concentration,
the standard curve having been established using blood samples, the fibrinogen concentration of which has been determined and the value of the property (Vp_{(TA)}) of which has been determined by steps a)-d) of the method according to the invention.

The level of D-dimers adjusted as a function of inflammation, Ddi_{S/I}, is thus expressed in initial fibrinogen equivalent units (FEUs).

Step e′2). Step e′2) consists in calculating the level of D-dimers which result from intravascular fibrin degradation (R) by correcting the level Ddi_{S/I} obtained in e′ 1) for the low levels of D-dimers (<4 pig/ml). This correction of the low levels of D-dimers as a function of fibrinogen has the objective of bringing the ratio Ddi_{S/I} to the threshold of 0.5 μg/ml FEUs (fibrinogen equivalent units).

The level R is calculated using the equation:

R=Ddi_{S/I}+[0.5−F_{Ddi-S}]

wherein F_{Ddi-S} is a correction factor for the low levels of D-dimers (<4 μg/ml), the value of which corresponds to the value of the correction factor, F, for the level of D-dimers of the sample (Ddi_{S}) on the standard curve having the equation:

y=ax²+bx+c

wherein y is the correction factor, F,
x is the level of D-dimers,
a, b and c are constants for the polynomial equation which links the correction factor and the level of D-dimers,
the standard curve having been established for blood samples, the level of D-dimers of which has been determined and for which the correction factor has been determined empirically so that the ratio Ddi_{S/I} is related back to the threshold of 0.5 FEUs (fibrinogen equivalent units).

The standard curve which was established by the present inventors is presented in FIG. 14(B).

Classification of the sample. Step e′) also comprises a step consisting in classifying the sample as a function of inflammation. More specifically, this step consists in calculating the ratio 1/[Fib]_{(Vp(TA))}, wherein [Fib]_{(Vp(TA))} is the fibrinogen concentration deduced in e′1), and classifying the sample from the patient:
in group I (patient without inflammation) if the ratio 1/[Fib]_{(Vp(TA))}>0.20 (that is to say if [Fib]_{(Vp(TA))}<5 g/1), or
in group II (patient with inflammation) if the ratio 1/[Fib]_{(Vp(TA))}≤0.20 (that is to say if [Fib]_{(Vp(TA))}≥5 g/1).

Step f′)

Step f′) of the second version of the method according to the invention consists in comparing the level of D-dimers resulting from fibrin degradation R obtained in e′), with respect to a threshold.

A level of D-dimers resulting from fibrin degradation, R, obtained in e′) below the threshold (preferably below the threshold of 0.5 μg/ml) makes it possible to exclude a thrombosis in the patient. A level of D-dimers resulting from fibrin degradation, R, obtained in e′) above the threshold (preferably above the threshold of 0.5 μg/ml) is indicative of the possibility of a thrombosis in the patient.

Step g′)

Step g′) is based on the fact that, in hyperfibrinolysis, fibrinogen is degraded by plasmin generated in great excess. Fibrin degradation products (FDPs), including the D-dimers, along with fibrinogen degradation productions (FgDPs) appear in the circulation, as shown in FIG. 2. Thus, high levels of D-dimers are generated in cancer and in significant coagulation activation states, in combination with high levels of FgDPs. It is thus possible to measure, without implied distinction, the level of FgDPs generated by hyperfibrinolysis or the level of D-dimers generated by hyperfibrinolysis.

The method according to the invention therefore comprises a step g′) of calculating the level of D-dimers generated by hyperfibrinolysis present in coagulation activation states using the level of D-dimers in the sample (Ddi_{S}) and the level of D-dimers resulting from fibrin degradation (R) obtained in e′) or using the level of D-dimers adjusted as a function of inflammation, Ddi_{S/I}, obtained in e1) and the level R obtained in e′).

More specifically, step g′) consists in:

g′1) calculating the level of D-dimers generated by hyperfibrinolysis, Ddi_{HF} or (R/Ddi_{S})ₚₐₜᵢₑₙₜ, as the ratio between the adjusted and corrected level of D-dimers (R) determined in step e′) and the level of D-dimers in the sample (Ddi_{S}), using the equation:

$${Ddi}_{HF} = {( R / {Ddi}_{S} )}_{patient} = \frac{R}{{Ddi}_{S}}$$

g′2) calculating the level of D-dimers generated by hyperfibrinolysis, Ddi_{HF} or (R/Ddi_{S/I})ₚₐₜᵢₑₙₜ, as the ratio between the adjusted and corrected level of D-dimers (R) determined in step e′) and the level of D-dimers adjusted as a function of inflammation, Ddi_{S/I}, obtained in e′1), using the equation:

$${Ddi}_{HF} = {( R / {Ddi}_{S / I} )}_{patient} = \frac{R}{{Ddi}_{S / I}}$$

Step h′)

Finally, step h′) of the second version of the method according to the invention comprises comparing the level of D-dimers generated by hyperfibrinolysis (Ddi_{S}) obtained in g′) with respect to a threshold, and comparing the ratio TA/FFT with respect to a threshold. The replacing of FFT, used in step h), with the ratio TA/FFT, used in step h′), makes it possible to differentiate a thrombosis, for which the time to reach the fibrin clot polymerization plateau is more rapid, from a coagulation activation state and from a thrombophilia.

More specifically, step h′) comprises first of all steps consisting in:

h′1) determining, for the level Ddi_{S} of the sample, the value of the ratio (R/Ddi_{S})_{standard} on the standard curve having the equation:

y=ax^{-b}

wherein x is the level of D-dimers,
y is the ratio between the level of D-dimers which result from intravascular fibrin degradation and the level of D-dimers, (R/Ddi_{S}),
a and b are the constants for the equation which links the ratio R/Ddi_{S} and the level of D-dimers,
the standard curve having been established:
using, if the sample from the patient has been classified in group I: blood samples classified in group I by step e′) of the method of the invention, the level of D-dimers of which is known or has been determined, and the level R of which has been determined by steps a)-e2) of the method according to the invention; and
using, if the sample from the patient has been classified in group II: blood samples classified in group II by step e′) of the method of the invention, the level of D-dimers of which is known or has been determined and the level R of which has been obtained by steps a)-e′2) of the method according to the invention;

h″1) comparing the value of the level of D-dimers generated by hyperfibrinolysis (R/Ddi_{S})ₚₐₜᵢₑₙₜ obtained in g′1) with the value of the ratio (R/Ddi_{S})_{standard} obtained in h′1), wherein (R/Ddi_{S})ₚₐₜᵢₑₙₜ below the ratio standard (R/Ddi_{S}) makes it possible to exclude thrombosis in the patient, and wherein (R/Ddi_{S})ₚₐₜᵢₑₙₜ above or equal to the ratio (R/Ddi_{S})_{standard} is indicative of a possibility of thrombosis in the patient;

h′2) determining, for the level Ddi_{S} of the sample, the value of the ratio (R/Ddi_{S/I})_{standard} on the standard curve having the equation:

y=ax^{-b}

wherein x is the level of D-dimers,
y is the ratio between the level of D-dimers which result from intravascular fibrin degradation and the level of D-dimers adjusted as a function of inflammation (R/Ddi_{S/I}), a and b are the constants for the equation which links the ratio R/Ddi_{S/I} and the level of D-dimers,
the standard curve having been established:
using, if the sample from the patient has been classified in group I: blood samples classified in group I by step e′) of the method of the invention and the level of D-dimers of which is known or has been determined, the level Ddi_{S/I} of which has been obtained by steps a)-e′1) of the method according to the invention, and the level R has been obtained by steps a)-e′2) of the method according to the invention; and
using, if the sample from the patient has been classified in group II: blood samples classified in group II by step e′) of the method of the invention and the level of D-dimers of which is known or has been determined, the level Ddi_{S/I} of which has been obtained by steps a)-e′1) of the method according to the invention, and the level R of which has been obtained by steps a)-e′2) of the method according to the invention;

h″2) comparing the value of the level of D-dimers generated by hyperfibrinolysis (R/Ddi_{S/I})ₚₐₜᵢₑₙₜ obtained in g′2) with the value of the ratio (R/Ddi_{S/I})_{standard} obtained in h′2), wherein (R/Ddi_{S/I})ₚₐₜᵢₑₙₜ below the ratio (R/Ddi_{S/I})_{standard} makes it possible to exclude thrombosis in the patient, and wherein (R/Ddi_{S/I})ₚₐₜᵢₑₙₜ above or equal to the ratio (R/Ddi_{S/I})_{standard} is indicative of a possibility of thrombosis in the patient.

Step h′) further comprises a step which consists in:

h′3) calculating the ratio TA/FFT wherein TA is the time to reach the fibrin polymerization plateau determined in step d) and FFT is the fibrin clot formation time determined in step d); and
h″3) if the sample from the patient has been classified in group I: comparing the ratio TA/FFT with respect to a first threshold, in particular to a first threshold of 1.75 for a Control Time of 135 seconds, wherein:
a ratio TA/FFT above the first threshold makes it possible to exclude thrombosis and to diagnose a thrombophilia or a coagulation activation state in the patient classified in group I, and
a ratio TA/FFT below or equal to the first threshold is indicative of a thrombosis in the patient classified in group I;
   if the sample from the patient has been classified in group II: comparing the ratio TA/FFT with respect to a second threshold, in particular to a second threshold of 1.65 for a Control Time of 135 seconds, wherein:
a ratio TA/FFT above the second threshold makes it possible to exclude thrombosis and to diagnose a thrombophilia or a coagulation activation state in the patient classified in group II, and
a ratio TA/FFT below or equal to the second threshold is indicative of a thrombosis in the patient classified in group II,
   wherein the Control Time is the average fibrin clot formation time of samples from healthy subjects with no suspicion of thrombosis, measured according to steps a)-d).

On the basis of the samples tested, the inventors have determined a first threshold of 1.75 for the samples classified in group I in step e′) and a second threshold of 1.55 for the samples classified in group II in step e′). Those skilled in the art will understand that these thresholds are predetermined and that they can vary as a function of the total number of samples tested or of the batches of reagents used.

It is possible to distinguish a thrombosis (for which the ratio TA/FFT is below or equal to the threshold) from a coagulation activation state and from a thrombosis with thrombophilia (for which the ratio TA/FFT is above the threshold) by considering the fibrin clot formation time (FFT). Indeed, the FFT is always short (≤[Control Time 1 standard deviation], for example <120 seconds for a Control Time at 135 seconds) in thrombophilia, whereas it is equal to the Control Time in coagulation activation states.

Preferably, high levels of D-dimers specific for venous thrombosis obtained at the end of the method are representative of the extent of the pulmonary embolism (PE) or of the deep vein thrombosis (DVT). Thus, in the case where a thrombosis is diagnosed in the patient by a method according to the invention, the level of D-dimers, R, obtained in step e′), related back to a clinically used scale, that is to say with respect to the threshold of 0.5 pig/ml, is proportional to the extent, of the PE or of the DVT, and therefore to the seriousness of the disease. Indeed, the diagnosis of seriousness is usually determined in imaging by the number and type of pulmonary arteries affected.

More specifically, in the case where a thrombosis is diagnosed in the patient in step h″3) of a method according to the invention, the level of D-dimers specific for venous thromboembolism (Ddi_{VTE}) is the level of D-dimers, R, obtained in step e′).

In the case where a thrombosis is excluded in steps h″1), h″2) and h″3), but the level R obtained in step e′) is above the threshold, and preferably above the threshold of 0.5 μg/ml, the method also comprises a step consisting in calculating the level of D-dimers specific for venous thromboembolism (Ddi_{VTE}) using the following equation:

$${Ddi}_{VTE} = 0.5 \times \frac{R}{{Ddi}_{S}}$$

wherein Ddi_{S} is the level of D-dimers in the sample. This calculation makes it possible to relate the level of D-dimers specific for venous thromboembolism back to a level below the threshold.

The levels of D-dimers specific for venous thrombosis thus provided at the end of the method are then representative of the extent of the pulmonary embolism or of the deep vein thrombosis.

### C. Automation

The measurement of optical density of step c) can be carried out by any suitable method known in the art. For example, the OD measurement of step c) can be carried out using any suitable existing instrument, and in particular a turbidimeter or a spectrophotometer. In particular, in some embodiments, at least steps c) and d) of the method according to the invention are carried out on a routine automated diagnostic device, preferably a coagulation analyzer. Preferentially, all the steps of the method according to the invention are carried out on such an automated device. More preferentially, this measurement is carried out at the same time as the usual assaying of the D-dimers on the routine automated device. For example, as described in examples 9 and 10, the method according to the invention is carried out on the STA-R® Max or STA-R® Evolution Expert Series automated devices from the Stago group.

Such an automated device makes it possible to simultaneously load the samples, to perform the mixing operations and the incubation, to measure the optical densities at a wavelength and to determine the clot formation profile obtained with a single sample in an emergency situation, or several samples simultaneously. The whole process is carried out in less than 10 minutes. This makes it a method which is fast, reliable and reproducible in a patient.

A method according to the invention can also be used on a remote biology device or a portable device at the bedside of the patient. In some preferred embodiments, the remote biology device or the portable device used is based on a physical method, preferably an optical method, such as a thromboelastography or rheometry method or an image analysis method. Preferentially, all the steps of this variant of the method according to the invention are carried out on such a device, in order to determine the fibrin clot formation profile obtained with a single sample in an emergency situation. The whole process is carried out in less than 5 minutes using the same tube of blood as for the usual assaying of the D-dimers.

### II—In Vitro Methods of Diagnosis

The method for assaying D-dimers specific for venous thromboembolism according to the invention makes it possible, using a blood sample (for example of plasma or whole blood) from patients, to diagnose venous thromboembolism in these patients, regardless of their age and regardless of their underlying pathological or physiological condition. As shown by the results obtained by the present inventors, a method according to the invention makes it possible to diagnose 100% of patients suffering from thromboembolism without false negatives, and makes it possible to exclude more patients than with the usual method for assaying D-dimers.

Thus, the present invention relates to a method for in vitro diagnosis of venous thromboembolism (VTE) in a patient, comprising steps consisting in:
carrying out a quantitative assay of D-dimers specific for VTE in a blood sample from the patient according to the first or the second version of the method described herein in order to obtain a level of D-dimers specific for VTE in the sample, and
providing a diagnosis regarding the patient.

Generally, the second version of the method for assaying D-dimers specific for VTE according to the invention will preferably be chosen in the case where the patient to be tested is an elderly individual, a patient suffering from cancer, a patient suffering from an infection or a patient suffering from thrombophilia. In the other cases, the first version of the method according to the invention may be used.

If the first version of the method is used, the diagnosis regarding the patient may be (i) exclusion of thrombosis, (ii) acute coagulation activation state, or (iii) thrombosis. If the second version of the method is used, the diagnosis regarding the patient may be (i) exclusion of thrombosis, (ii) acute coagulation activation state, (iii) thrombosis in a patient with thrombophilia, or (iv) thrombosis, including thrombosis with a low level of seriousness or thrombosis in the event of pulmonary infarction.

Unless otherwise defined, all the technical and scientific terms used in the description have the same meaning as the term commonly understood by an ordinary specialist in the field to which this invention belongs. Likewise, all the publications, patent applications, all the patents and all other references mentioned herein are incorporated by way of reference.

### EXAMPLES

The following examples describe some embodiments of the present invention. However, it is understood that the examples are presented merely by way of illustration only and do not in any way limit the scope of the invention.

### FIGURE LEGENDS

FIG. 1: Diagram of coagulation activation. FpA-FpB=fibrinopeptides A and B; TAT=thrombin-antithrombin complex; and F1+2=prothrombin fragments.

FIG. 2: Dynamics of D-dimer formation by hypercoagulation, inflammation, and hyperfibrinolysis in coagulation activation states and venous thromboembolism (VTE).

FIG. 3: Principle of the method for assaying D-dimers specific for VTE according to the invention, in a plasma or whole-blood sample, comprising the usual assaying of D-dimers of the sample and the dynamic measurement of the fibrin formation of this sample.

FIG. 4: Interpretation of the method for assaying D-dimers specific for VTE, in a biological sample, on the basis of the D-dimers adjusted as a function of the D-dimers generated by hypercoagulation, inflammation and hyperfibrinolysis in said sample from the patient according to the first version of the method.

FIG. 5: Fibrin clot formation profile (step d), (A) in the case of a plasma from a normal patient (PN) in which the fibrinogen is 3.5 g/l and of a sample of plasma from a patient with a hypercoagulation state (P Hyper), in which the fibrinogen is 3.0 g/l, by measurement of the optical density (OD) as a function of time, according to the method of the invention; and (B) in the case of a whole-blood sample from a normal patient (PN) in which the fibrinogen level is 2.0 g/l and of a whole-blood sample from a hyperfibrinogenemic patient (P Hyperfib) in which the fibrinogen level is 5.0 g/1, by thromboelastometric measurement of the amplitude at the time to reach the plateau as a function of time, according to a variant of the method of the invention.

FIG. 6: Variation in optical density (DOD) at the time to reach the fibrin polymerization plateau, by the method according to the invention, for the 51 plasma samples from patients with a suspicion of thrombosis, as a function of inflammation, represented (A) by the level of C-reactive protein (CRP); and (B) by the level of fibrinogen (Fib).

FIG. 7: Time to reach the plateau as a function of fibrin formation time, by the method according to the invention (A) for 219 samples from patients with or without known cancer, or with a suspicion of thrombosis; and (B) for 34 samples from patients suffering from cancer, with a suspicion of thrombosis.

FIG. 8: Level of D-dimers generated by hypercoagulation as a function of D-dimers of the sample (step e)) (A) for 87 samples from patients with a suspicion of thrombosis; and (B) for 28 samples from patients with an active cancer.

FIG. 9: Variations in optical density (DOD) at the time to reach the fibrin polymerization plateau, measured according to the method of the invention, as a function of the level of fibrinogen determined by the reference method (Clauss) (A) in 219 samples from patients with or without cancer, with a suspicion of thrombosis; and (B) in 23 samples from patients suffering from active cancer, with a suspicion of thrombosis. (C) Amplitude at the time to reach the fibrin polymerization plateau measured according to the variant of the method according to the invention in 7 whole-blood samples from patients with a suspicion of thrombosis, as a function of the level of fibrinogen determined in plasma by the Clauss method.

FIG. 10: Correlation in the samples from 14 patients with and without venous thrombosis, between the level of fibrinogen degradation products (FgDPs) measured by the STA Liatest® FDP latex method (Stago) and the level (A) of the usual D-dimers (Stago method); (B) of the D-dimers (Stago method) adjusted as a function of the D-dimers generated by hypercoagulation and inflammation, according to the method of the invention carried out on plasma; and (C) of the D-dimers (Stago method) adjusted as a function of the D-dimers generated by hypercoagulation and inflammation, according to a variant of the method of the invention carried out on whole-blood samples.

FIG. 11: Correlation in the samples from 14 patients with and without venous thrombosis, between the level of fibrinogen degradation products (FgDPs) measured by the STA Liatest® FDP latex method (Stago) and the level (A) of the D-dimers (method of the Instrumentation Laboratory IL); and (B) of the D-dimers (IL method) adjusted as a function of the D-dimers generated by hypercoagulation and inflammation, according to the method of the invention carried out on plasma samples.

FIG. 12: Comparison of the adjustment of D-dimers (Stago method), by the method of the invention and by the ratio [D-dimers/Fibrinogen] (A) in the plasmas from 215 patients without venous thrombosis in imaging; and (B) in the plasmas from 21 patients with PE and/or DVT in imaging. (I: inflammation, F: hyperfibrinolysis, Ddi: D-dimers, Fib: fibrinogen).

FIG. 13: Comparison of the adjustment of D-dimers (IL method), by the method of the invention and by the ratio [D-dimers/Fibrinogen] (A) in the plasmas from 199 patients without venous thrombosis in imaging; and (B) in the plasmas from 15 patients with PE and/or DVT in imaging. (I: inflammation, F: hyperfibrinolysis, Ddi: D-dimers, Fib: fibrinogen).

FIG. 14: Correlation between the correction factor F_{Ddi-S} for the low levels of D-dimers (<4 μg/ml) (A) and the level of D-dimers, R, resulting from intravascular fibrin degradation (B) obtained after adjustment as a function of inflammation (Ddi_{S/I}) and correction for the low levels of D-dimers (<4 μg/ml) by the equation R=Ddi_{S/I}+[0.5−F_{Ddi-s}] as a function of the level of D-dimers of the sample (Ddi_{S}), according to the method of the invention carried out on plasma samples.

FIG. 15: Comparison of the percentage exclusion of thrombosis as a function of age, by i) the assaying of D-dimers, ii) the assaying of D-dimers adjusted with respect to age, iii) the method of the invention (A) for 796 patients with a suspicion of venous thrombosis; and (B) for the 58 patients suffering from cancer among the 796 patients with a suspicions of venous thrombosis.

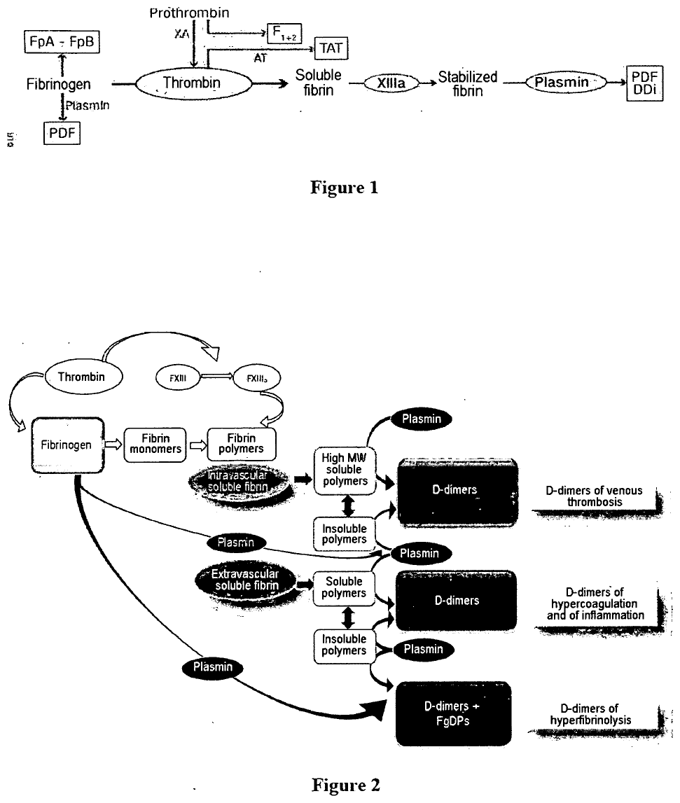

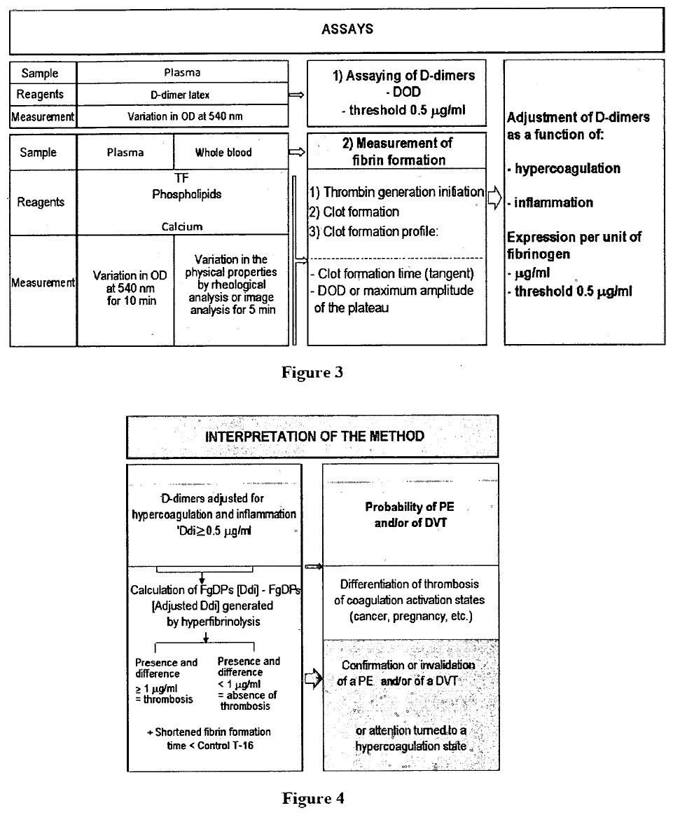

### EXPERIMENTAL PROTOCOLS

The protocols used in the examples are the following:

### Protocol A

Protocol A is a method carried out on the STAR® Evolution Expert Series automatic coagulation analyzer (Stago).

The following are simultaneously added, by the instrument, to 8 cuvettes of the STAR® Evolution automated device (step a)):
200 μl of pure plasma sample, obtained after centrifugation of the citrated tube intended for usual assaying of D-dimers, for 15 minutes, at a speed of 2500 g,
50 μl of a mixture of tissue factor and of phospholipids [TF+PL], that has been freeze-dried and taken up with water; at final concentrations of tissue factor (TF) of 2 to 5 pM and of phospholipids (PL) of 4 μM.

The automated device stirs by means of the arm and carries out an incubation for 300 seconds at 37° C. It then adds 50 μl of CaCl₂ at a final concentration of 16.7 mM, and stirs, by means of the needle, the triggering reagent (step b)). The automated device then measures the optical density (OD) at 540 nm as a function of the time for 10 minutes (step c)) and plots the fibrin formation curve.

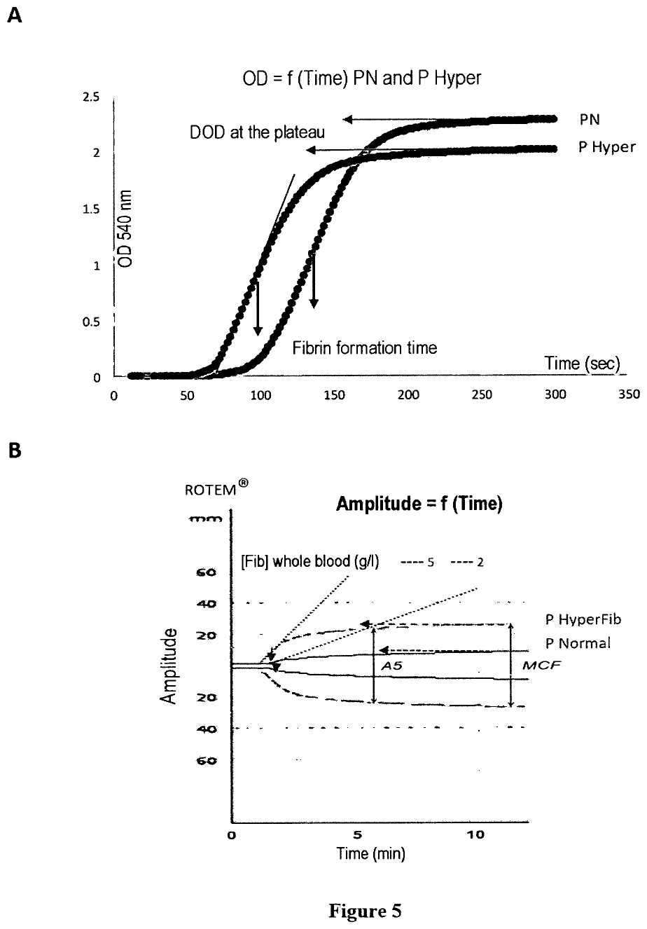

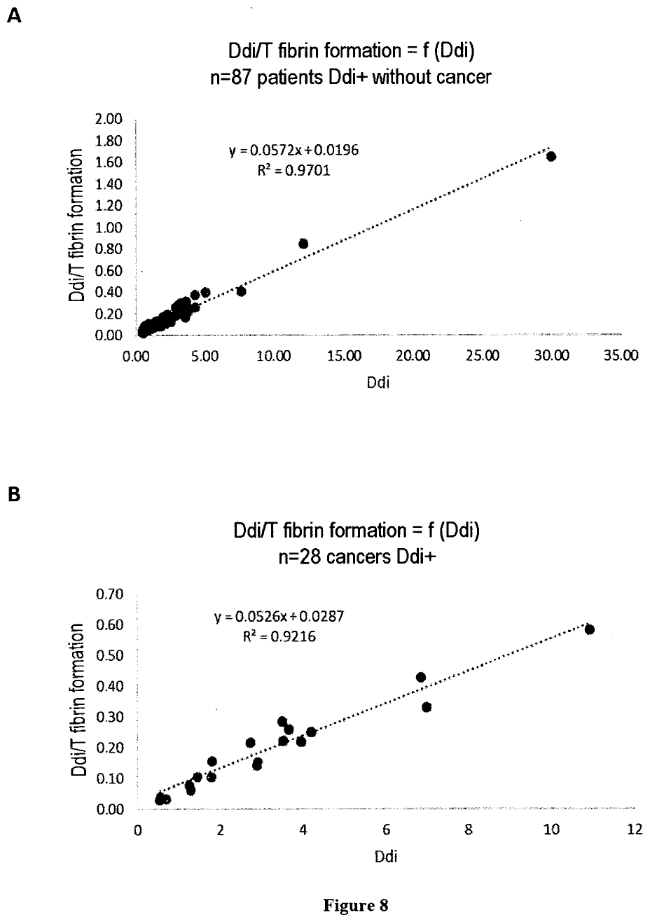

The algorithm for “post-processing” of the measurement makes it possible to calculate the fibrin formation time (FFT) from the tangent to the curve, and the variation in OD (DOD) at the time to reach (TA) the plateau, from the OD at the time TA of the plateau and the OD at the time T0 to 10 seconds, as described in FIG. 5a (step d)). The level of D-dimers adjusted as a function of the D-dimers generated by hypercoagulation is calculated by the formula [(X)×(FFT/Control Time)], wherein X is the level of D-dimers measured, as shown in FIG. 8 and example 3.

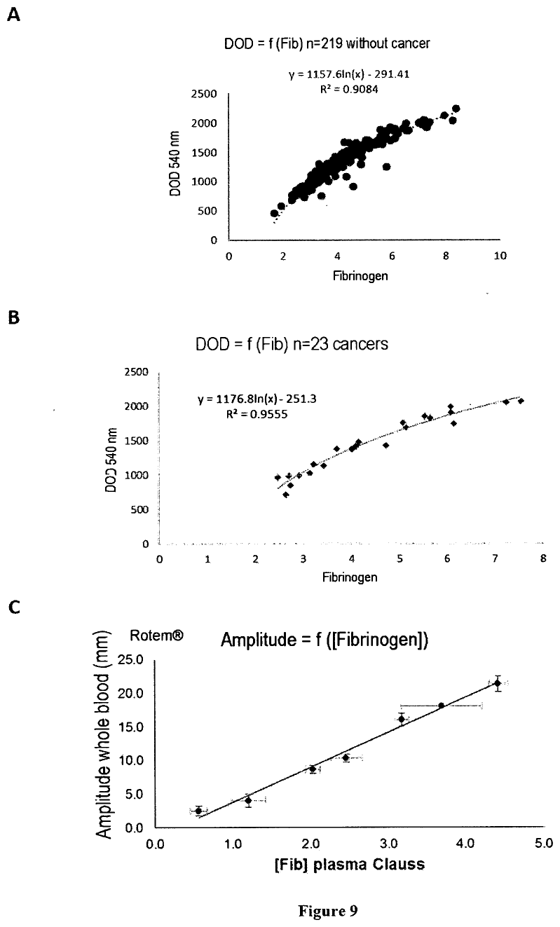

The level of D-dimers adjusted as a function of the D-dimers generated by inflammation is calculated by the ratio [adjusted D-dimers/inflammation]. The level of inflammation is determined from the equation y=a ln(x)−b, wherein y is the DOD, x is the fibrinogen concentration, and a and b are the constants for the logarithmic equation which links the level of the fibrin plateau and the fibrinogen concentration, as shown in FIG. 9 and example 4. The D-dimers are thus expressed in fibrinogen equivalent units (FEUs) (step e)).

The time to reach the clot polymerization is calculated by the ratio TA/FFT, wherein TA is the time to reach the fibrin polymerization plateau determined in step d) and FFT is the fibrin clot formation time determined in step d).

### Protocol B

Protocol B is a method carried out on the Rotem® delta automatic coagulation analyzer.

300 μl of non-diluted whole blood (steps a) and b) of the variant of the method according to the invention), taken from the citrated tube intended for the usual assaying of D-dimers, preheated to 37° C., are added to a cuvette of the instrument, preheated beforehand, containing 40 μl of a mixture of tissue factor and of phospholipids [TF+PL], that is freeze-dried and taken up with water; at final concentrations of tissue factor (TF) of 2 to 5 pM and of phospholipids (PL) of 4 μM, and of CaCl₂ at a final concentration of 16.7 mM. The thromboelastomeric measurement is initiated (step c) of the variant of the method according to the invention) then 300 μl of the mixture of blood and reagent are analyzed by the instrument which continuously records, for 30 minutes, the coagulation activation, the formation, the polymerization and the stability of the fibrin clot, and provides the usual parameters (CT, CFT, angle α, A5, A10, MCF, MCF-t).

The fibrin formation time (FFT) from the tangent and the amplitude at the time to reach (TA) the plateau of the fibrin formation curve are calculated from the temporal change in the amplitude as a function of time, as described in FIG. 5(B) (step d) of the variant of the method according to the invention). The level of D-dimers adjusted as a function of the D-dimers generated by hypercoagulation is calculated by the formula [(X)×(FFT/Control Time)], wherein X is the level of D-dimers measured, as shown in example 3.

The level of D-dimers adjusted as a function of the D-dimers generated by inflammation is calculated by the ratio [adjusted D-dimers/level of inflammation]. This level is determined from the linear equation which links the amplitude at the time to reach the plateau (mm) and the fibrinogen concentration, as shown in FIG. 9 and example 4. The D-dimers are thus expressed in initial fibrinogen equivalent units (FEUs) (step e) of the variant of the method according to the invention).

### Protocol C

Protocol C is a method carried out on the STAR® Evolution Expert Series automatic coagulation analyzer (Stago).

The following are simultaneously added to the cuvettes of the STAR® Evolution automated device, by the instrument (step a)):
200 μl of sample of each of the negative control or positive control plasmas, freeze-dried and reconstituted with water,
50 μl of a mixture of tissue factor and of phospholipids [TF+PL], that has been freeze-dried and taken up with water; at final concentrations of tissue factor (TF) of 2 pM and of phospholipids (PL) of 4 μM.

The automated device stirs by means of the arm, carries out an incubation for 300 seconds at 37° C. then adds 50 μl of CaCl₂ at a final concentration of 16.7 mM, and stirs, by means of the needle, the triggering reagent (step b)). The automated device then measures the optical density (OD) at 540 nm as a function of the time for 10 minutes (step c)) and the fibrin formation curve is plotted for each of the control plasmas.

The algorithm for “post-processing” of the measurement makes it possible to calculate the fibrin formation time (FFT) from the tangent to the curve, and the variation in OD (DOD) at the time to reach (TA) the plateau, from the OD at the time T of the plateau and from the OD at the time T0 to 10 seconds for each of the control plasmas, as described in FIG. 5(A) (step d))).

The level of D-dimers adjusted as a function of the D-dimers generated by hypercoagulation is calculated by the formula [(X)×(FFT/Control Time)], wherein X is the level of D-dimers measured, as shown in FIG. 8 and example 3.

The level of D-dimers adjusted as a function of the D-dimers generated by inflammation is calculated by the ratio [adjusted D-dimers/level of inflammation]. This level is determined from the equation y=a ln(x)−b, wherein y is the DOD, x is the fibrinogen concentration, a and b of the constants for the logarithmic equation which links the level of the fibrin plateau and the fibrinogen concentration, as shown in FIGS. 9(A) and (B) and in example 4. The D-dimers are thus expressed in fibrinogen equivalent units (FEUs) (step e)).

### Example 1: Fibrin Formation Time of Various Plasmas from Normal Patients and from Patients with a Suspicion of Thrombosis (with or without Thrombosis, and with or without Cancer)

The protocol used in this example is protocol A. Step d) of the method used in this example is without implied distinction that of the first or the second version of the method.

Among the samples from patients with a suspicion of PE and/or of DVT that are tested, the samples from patients with a coagulation activation state are differentiated from the normal patients (normal healthy subjects, with no suspicion of thrombosis), from those with an exclusion of thrombosis, and from those with a positive diagnosis of venous thrombosis in imaging, with regard to the fibrin formation time (FFT), representing hypercoagulation, with regard to the time to reach (TA) the plateau, and with regard the time to reach clot polymerization (ratio TA/FFT), as shown in FIG. 5 and table 1 below.

The fibrin formation time (FFT) is shorter for the patients exhibiting a hypercoagulation state; but surprisingly, there is no hypercoagulation state in the patients with a diagnosis of PE and/of DVT, contrary to the patients with an exclusion of thrombosis, including in cancer. Advantage is taken of this in the first version of the method of the invention in order to discriminate the patients with and without thrombosis.

The time to reach the plateau does not exceed 8 minutes, all patients included, except for the patients receiving anticoagulant treatment, for whom the rendering of the result is limited to the fibrin formation time.

The time to reach clot polymerization is shorter (ratio TA/FFT lower) in the patients with a diagnosis of PE and/or of DVT, than in the normal patients, or the patients with a coagulation activation state, or with an exclusion of thrombosis, including in cancer. Advantage is taken of this in the second version of the method of the invention in order to discriminate the patients with and without thrombosis.

TABLE 1
Discrimination of the plasmas from patients with a hypercoagulation state
among the normal patients and the patients with a suspicion of thrombosis,
whether or not they have a thrombosis or a cancer, with regard to the fibrin
formation time (FFT) and with regard to the time to reach (TA) the plateau
and with regard to the time to reach clot polymerization (TA/FFT).

| | Diagnosis | | | | | | | |
|---|---|---|---|---|---|---|---|---|
| | D-dimers (threshold | | | | | | | |
| Plasmas | 0.5 μg/ml) and/or | | | FFT (sec) | | TA (sec) | | TA/FFT |
| from | imaging | | N | Mean | Range | Mean | Range | Mean |
| Normal | Ddi negative | Negative | 150 | 133 | 88-206 | 1.82 | 172-324 | 1.82 |
| patients | | | | | | | | |
| Patients | Ddi negative | Negative | 127 | 141 | 84-304 | 1.62 | 154-440 | 1.62 |
| with | Ddi positive | Negative | 88 | 154 | 88-258 | 1.55 | 154-384 | 1.55 |
| suspicion of | without | | | | | | | |
| thrombosis | thrombosis | | | | | | | |
| without | Ddi positive | Positive | 21 | 166 | 116-218 | 1.45 | 176-298 | 1.45 |
| cancer | with | PE | | | | | | |
| | thrombosis | and/or | | | | | | |
| | | DVT | | | | | | |
| Patients | Imaging | Negative | 16 | 155 | 116-210 | 1.56 | 190-304 | 1.56 |
| with | without | | | | | | | |
| suspicion of | thrombosis | | | | | | | |
| thrombosis | Imaging | Positive | 6 | 176 | 126-212 | 1.46 | 204-298 | 1.46 |
| with cancer | with | PE | | | | | | |
| | thrombosis | and/or | | | | | | |
| | | DVT | | | | | | |

| | | | | | | |
|---|---|---|---|---|---|---|
| Under anticoagulants | 10 | 313 | 150-694 | 447 | 220-1122 | / |

### Example 2: Generation of D-Dimers by Hypercoagulation in Various Plasmas from Patients with a Suspicion of Thrombosis (with or without Thrombosis, and with or without Cancer)

The protocol used in this example is protocol A. Step e) of the method used in this example is that of the first version of the method.

Among the samples from patients with a suspicion of PE and/or of DVT that are tested, the D-dimers generated as a function of the fibrin formation time correlate well with the initial D-dimers of the sample, whether for the patients with a positive diagnosis of thrombosis in imaging or with an exclusion of thrombosis, with or without cancer, as shown by FIGS. 8 (A, B).

Advantage is taken of the linear function to adjust the D-dimers of the sample as a function of the D-dimers generated by hypercoagulation, by calculating the adjusted D-dimers Y, from the level of D-dimers of the sample X, by the following formula:

$$Y = X \times \frac{Fibrin formation time}{Control Time}$$

This makes it possible to amplify the D-dimers proportionally to the elongation of the fibrin formation time FFT in the patients with thrombosis, and to subtract them proportionally to the shortening of the fibrin formation time FFT in the patients with hypercoagulation.

The ratio varies as a function of the type and batch of D-dimer reagent used for the assay; and as a function of the batch of tissue factor-phospholipid reagent used for measuring the fibrin formation. The batches of reagents can then advantageously be calibrated according to this formula.

### Example 3: Adjustment of the D-Dimers as a Function of the D-Dimers Generated by Hypercoagulation, for Various Plasmas from Patients with a Suspicion of Thrombosis (with and without Thrombosis, and with or without Cancer)

The protocol used in this example is protocol A. Step e) of the method used in this example is that of a first version of the method.

Advantage is taken of the linear correlation y=ax+b of example 2 in the method according to the invention, in order to adjust the D-dimers as a function of the D-dimers generated by hypercoagulation, as described in particular in example 2 and table 2 below. The patients with and without thrombosis are especially discriminated on the basis of a high level of D-dimers 3.6 times higher for the patients with thrombosis than for the patients without thrombosis and 2.3 times higher for the cancer patients with a thrombosis than for the patients without thrombosis, as shown in table 2 below.

The adjustment of the D-dimers as a function of the D-dimers generated by hypercoagulation makes it possible to subtract the D-dimers generated by hypercoagulation in coagulation activation states.

TABLE 2
Adjustment of the D-dimers as a function of the D-dimers generated by
hypercoagulation in the plasmas from patients with a suspicion of thrombosis
(with or without thrombosis, and with or without cancer).

| | | | | | Level of adjusted | |
|---|---|---|---|---|---|---|
| | Diagnosis | | Level of initial D-dimers | | D-dimers/hypercoag | |
| | D-dimers and/or | | (μg/ml) | | (μg/ml) | |
| Plasmas from | imaging | Number | Mean | Range | Mean | Range |
| Normal | Ddi negative | 150 | 0.28 | 0.27-0.47 | 0.28 | 0.27-0.54 |
| patients | | | | | | |
| Patients with | Ddi negative | 127 | 0.31 | 0.27-0.49 | 0.33 | 0.19-0.66 |
| suspicion of | Ddi positive | 88 | 1.60 | 0.51-11.6 | 2.00 | 0.43-19.0 |
| thrombosis | without | | | | | |
| with or | thrombosis | | | | | |
| without cancer | Ddi positive | 21 | 5.72 | 1.33-30.0 | 7.19 | 1.41-41.0 |
| | with thrombosis | | | | | |
| Variation +/− thrombosis | | | ×3.6 | | ×3.6 | |
| Patients with | Imaging | 16 | 2.52 | 0.27-11.6 | 2.94 | 0.59-19.0 |
| cancer with | without | | | | | |
| suspicion of | thrombosis | | | | | |
| thrombosis | Imaging | 6 | 5.21 | 2.73-10.9 | 6.89 | 2.59-15.5 |
| with cancer | with thrombosis | | | | | |

| | | |
|---|---|---|
| Variation +/− thrombosis | ×2.1 | ×2.3 |

### Example 4: Level of the Fibrin Plateau, of Various Plasmas from Normal Patients and from Patients with a Suspicion of Thrombosis (with or without Thrombosis, and with or without Cancer), by the Method of the Invention

### Plasma Samples

The protocol used in the first part of this example is protocol A. Step d) of the method used in this example is without implied distinction that of the first or the second version of the method.

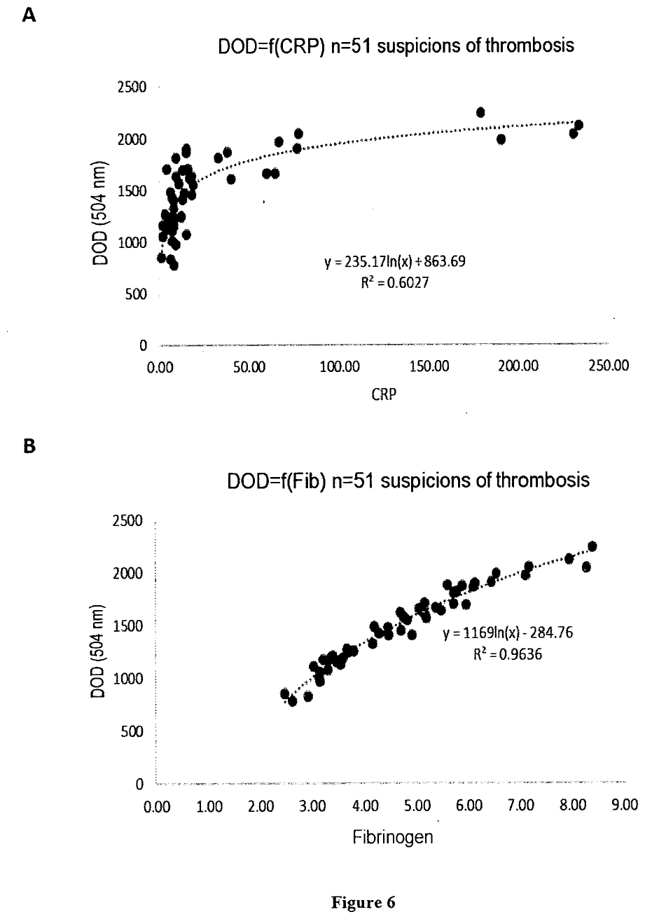

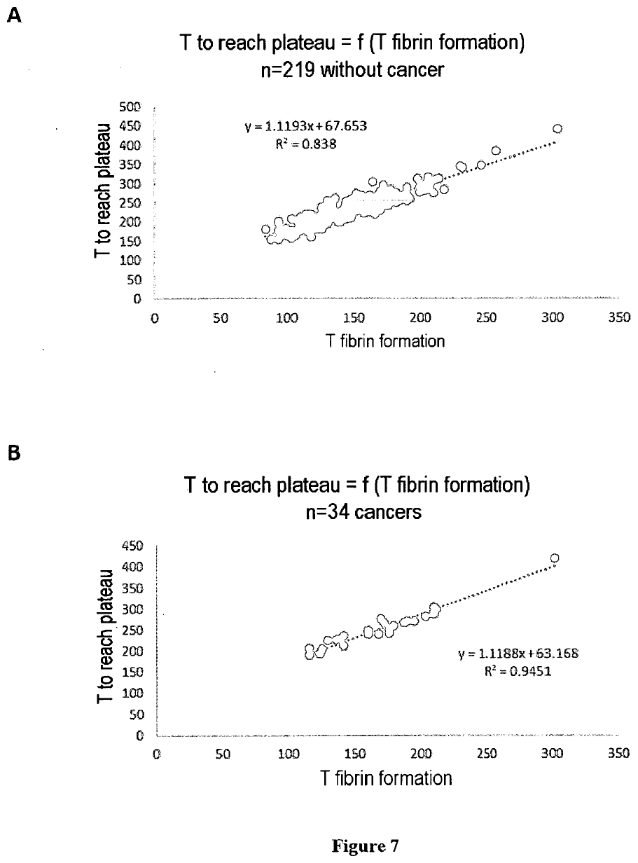

Among the samples from patients with a suspicion of PE and/or DVT that are tested, the samples from patients with a positive diagnosis of thrombosis in imaging or with an exclusion of thrombosis are differentiated from the patients with a coagulation activation state and the normal patients (normal healthy subjects with no suspicion of thrombosis) with regard to the level of the fibrin formation plateau, representative of inflammation, as shown in FIGS. 6 and 9, and table 3 below. The level of the DOD plateau by the method of the invention is higher the greater the inflammation, represented by C-reactive protein (CRP) and fibrinogen in FIGS. 6(A, B), independently of the presence or of the exclusion of thrombosis.

The level of the fibrin formation plateau, which represents the maximum clot polymerization, correlates well with the level of fibrinogen of the sample, regardless of the patients, according to a logarithmic equation y=a ln x+b, wherein y is the variation in optical density at the plateau start time, and ln x is the Naperian log of x which represents the level of fibrinogen, as described in FIGS. 9(A, B). The variable x is calculated from the equation, by the exponential function of the Naperian log of x. The factor linking the level of fibrinogen and the DOD is constant regardless of the patients, as shown in table 3 below.

The patients with and without thrombosis, including in the case of patients who have a cancer, are discriminated on the basis of the inflammation, represented by the amount of fibrinogen and/or of DOD, as shown in table 3 below.

TABLE 3
Level of the fibrin plateau of plasmas from patients with a suspicion of
thrombosis (with or without thrombosis, and with or without cancer) determined
by the DOD at the time to reach the fibrin formation plateau.

| | Diagnosis | | Level of fibrinogen | | | | |
|---|---|---|---|---|---|---|---|
| | D-di and/or | | (μg/ml) | | DOD 540 nm | | Delta |
| Plasmas from | imaging | Number | Mean | Range | Mean | Range | DOD/Fib |
| Normal | Ddi negative | 150 | 2.93 | 2.12-4.56 | 0.937 | 0.618-1.423 | 0.32 |
| patients | | | | | | | |
| Patients with | Ddi negative | 127 | 3.79 | 1.93-8.40 | 1.225 | 0.581-2.237 | 0.32 |
| suspicion of | Ddi positive | 88 | 4.50 | 1.67-8.27 | 1.437 | 0.458-2.119 | 0.32 |
| thrombosis | without | | | | | | |
| with or | thrombosis | | | | | | |
| without | Ddi positive | 21 | 4.83 | 3.00-7.54 | 1.509 | 1.007-2.059 | 0.31 |
| cancer | with | | | | | | |
| | thrombosis | | | | | | |
| Patients with | Imaging | 16 | 4.15 | 2.63-7.23 | 1.424 | 0.716-2.051 | 0.34 |
| suspicion of | without | | | | | | |
| thrombosis | thrombosis | | | | | | |
| with cancer | Imaging | 6 | 4.91 | 3.43-7.54 | 1.598 | 1.134-2.059 | 0.33 |
| | with | | | | | | |
| | thrombosis | | | | | | |

### Whole-Blood Samples

The protocol used in the second part of this example is protocol B. This method is carried out in the presence of an anti-platelet inhibitor, in the case in point cytochalasin D.

The level of inflammation is determined from the linear equation, which links the amplitude at the time to reach the plateau, according to the alternative method in whole blood as shown in FIG. 5(B), and the level of fibrinogen in Clauss, as shown in FIG. 9(C). The patients tested with the variant of the method according to the invention are discriminated both with the method based on the amplitude at the time to reach the plateau, and with the method of the invention based on the determination of DOD.

### Example 5: Adjustment of the D-Dimers as a Function of the D-Dimers Generated by Inflammation, for Various Plasmas from Normal Patients and from Patients with a Suspicion of Thrombosis (with or without Thrombosis, and with or without Cancer), by the Method of the Invention

The protocol used in this example is protocol A. Steps e) and f) of the method used in this example are without distinction those of the first or of the second version of the method.

Advantage is taken of the logarithmic function of example 4 in the method according to the invention, in order to adjust the D-dimers as a function of the D-dimers generated by inflammation, by the ratio R:

$$R = \frac{\begin{matrix}D \text{-} dimers adjusted as a function of the \\ D \text{-} dimers generated by hypercoagulation\end{matrix}}{Level of inflammation}$$

and to express them in fibrinogen equivalent units FEUs.

Since the amount of D-dimers generated by plasmin is approximately 50% of the FEU unit with the antibodies 8D2 and 2.1.16 of the latex reagent used, the positivity threshold of the measurement is thus 0.5 μg/ml.

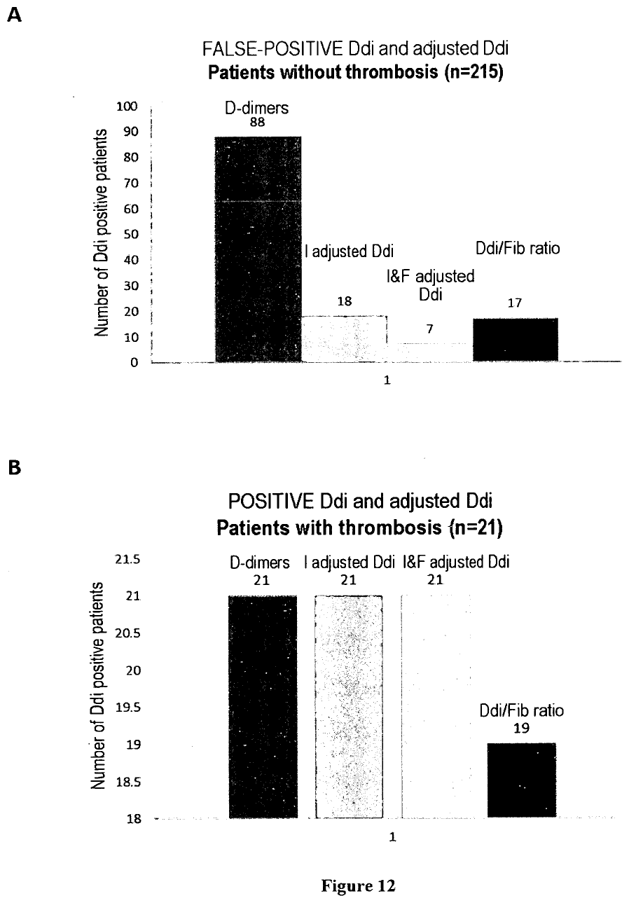

The level of adjusted D-dimers is determined with respect to a threshold, preferably a threshold of 0.5 μg/ml, in order to determine the probability of a pulmonary embolism (PE) or of a deep vein thrombosis (DVT) in the sample from the patient, as shown by FIGS. 12(A, B) and table 4 below.

The 127 patients excluded with regard to D-dimers all have a negative adjusted D-dimer level. Among the 88 patients without thrombosis, the D-dimers of whom are falsely positive, 80% have a negative adjusted D-dimer level and avoid imaging. More than 90% of the patients without thrombosis thus avoid imaging with the method of the invention.

100% of the patients with thrombosis have a positive adjusted D-dimer level ranging from 0.52 μg/ml to 10.5 μg/ml, as shown in table 4 below.

Among the samples from patients without thrombosis, found to be falsely positive in terms of adjusted D-dimers with a level of 0.50 μg/ml to 2.73 μg/ml, ⅓ have a level <0.60 μg/ml, ⅓ have a cancer, and ⅓ have a coagulation activation state.

TABLE 4
Discrimination of plasmas from patients with a suspicion of thrombosis
(with or without thrombosis, and with or without cancer), with an adjustment
of D-dimers as a function of the D-dimers generated by hypercoagulation
and as a function of the D-dimers adjusted for inflammation.

| | | | | | Level of adjusted | | |
|---|---|---|---|---|---|---|---|
| | Diagnosis | | Level of adjusted D- | | D-dimers/ | | |
| | Ddi and/or | | dimers/hypercoag H | | inflammation I | | Result |
| Plasmas from | imaging | N | Mean | Range | Mean | Range | rendered |
| Patients with a | Ddi negative | 127 | 0.33 | 0.19-0.66 | 0.09 | 0.04-0.21 | 0 False+ |
| suspicion of | Ddi positive | 88 | 2.00 | 0.43-19.0 | 0.42 | 0.11-2.73 | 18 False+ |
| thrombosis | without | | | | | | |
| with or | thrombosis | | | | | | |
| without | Ddi positive | 21 | 7.19 | 1.41-41.0 | 1.65 | 0.52-10.5 | 0 False− |
| cancer | with | | | | | | |
| | thrombosis | | | | | | |
| Patients | Imaging | 16 | 4.24 | 0.59-19.0 | 0.72 | 0.12-2.73 | 7 False+ |
| with a | without | | | | | | |
| suspicion of | thrombosis | | | | | | |
| thrombosis | Imaging | 6 | 7.20 | 2.59-15.5 | 1.44 | 0.58-3.65 | 0 False− |
| with cancer | with | | | | | | |
| | thrombosis | | | | | | |

### Example 6: Differentiation of D-Dimers Generated by Intravascular Fibrin, from Those Generated in High Coagulation Activation States with Regard to Hyperfibrinolysis

The protocol used in this example is protocol A. Step g) of the method used in this example is that of the first version of the method.

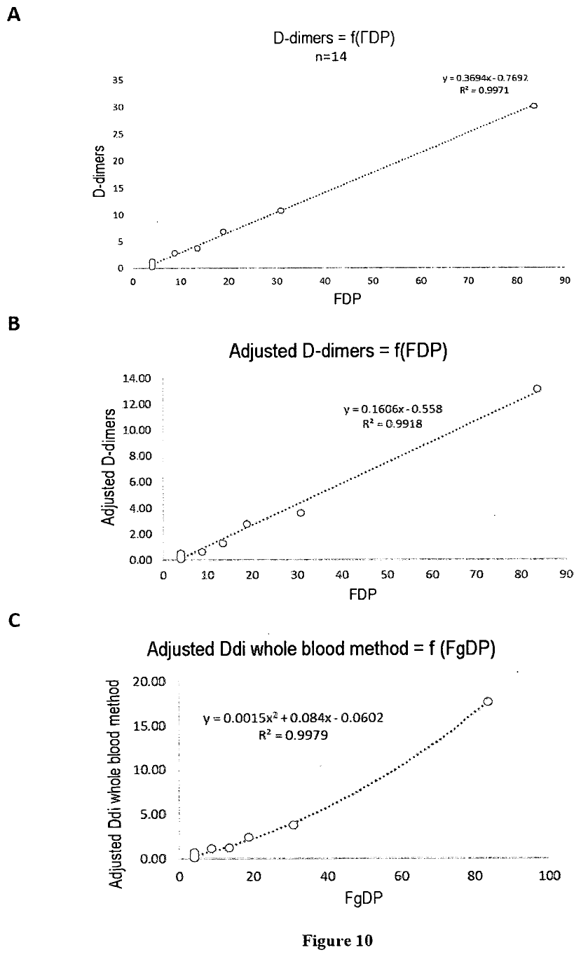

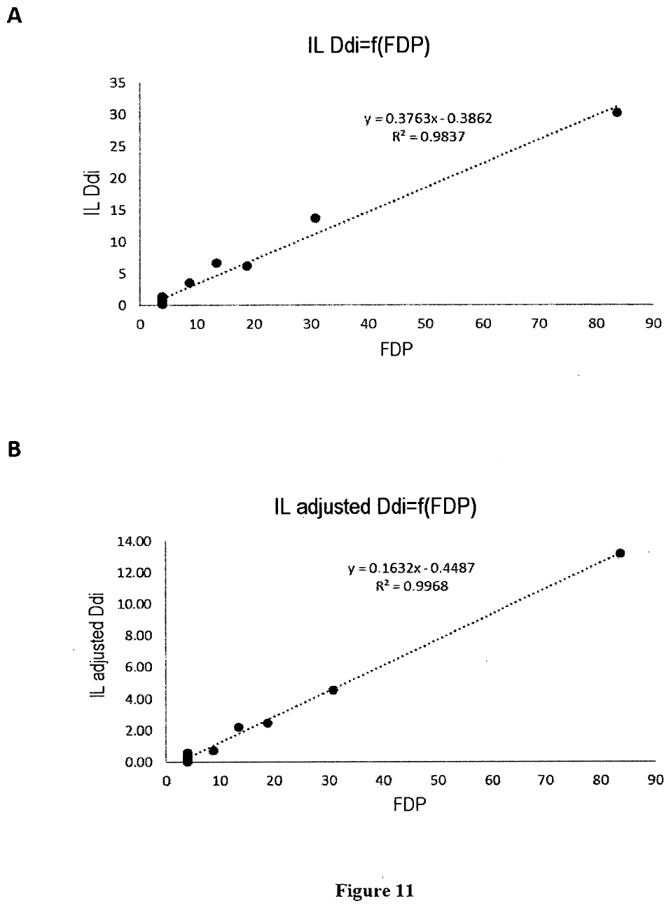

The differentiation between the D-dimers present in these activation states and those of thrombosis is carried out with regard to hyperfibrinolysis. The generation of fibrinogen degradation products FgDPs by plasmin in acute activation states enables their exclusion. The hyperfibrinolysis is calculated from the linear equations of FIG. 10(A, B) for the Stago reagent, and FIG. 11(A, B) for the IL reagent (step g)).

In the presence of hyperfibrinolysis (FgDP>7 μg/ml), the FgDPs generated with intravascular fibrin are systematically lower in the samples originating from patients with thrombosis than in those from patients without thrombosis; this being whatever the D-dimer reagent used. This makes it possible to discriminate the patients with thrombosis from those without thrombosis, on the basis of a difference between FgDPs before and after D-dimer adjustment which is positive, at the threshold of 1 μg/ml, as shown in table 5 below. Conversely, a difference which is negative or <1.0 in the patients without thrombosis makes it possible to turn attention to an acute coagulation activation state.

TABLE 5
Discrimination of the plasmas from patients with a suspicion of thrombosis (with
or without thrombosis, and with or without cancer), as a function of hyperfibrinolysis
with deduction of FgDPs, by the optical method on STAR ® (Stago).

| | | | | | | Difference | |
|---|---|---|---|---|---|---|---|
| | Diagnosis | | | | FgDP | FgDP | |
| Plasmas | Ddi positive | | Adjusted | | Adjusted | threshold ≥1 | Result |
| from | and imaging | N | Ddi | FgDP/Ddi | Ddi | μg/ml | rendered |
| Patients with | Ddi neg | 127 | 0.09 | Neg | Neg | / | Negative |
| suspicion of | Without | 69 | 0.26 | Neg | Neg | / | Negative |
| thrombosis | thrombosis | 4 | 0.51 | Neg | Neg | / | 4 False+ |
| with or | | 9 | 1.13 | 11.2 | 10.9 | +0.3 | Negative |
| without | | 6 | 0.91 | 15.3 | 9.2 | +6.1 | 6 False+ |
| cancer | | | | | | (+2.6 to +20.3) | |
| | With | 2 | 0.55 | Neg | Neg | / | Positive |
| | thrombosis | 19 | 1.77 | 18.8 | 14.5 | +4.3 | Positive |
| | | | (0.58-10.5) | (8.3-83.3) | (7.1-68.7) | (+1.2 to +14.6) | |
| Patients with | Without | 10 | 0.26 | Neg | Neg | / | Negative |
| suspicion of | thrombosis | 2 | 1.97 | 16.1 | 15.8 | +0.3 | Negative |
| thrombosis | | 1 | 0.51 | Neg | Neg | / | 1 False+ |
| with cancer | | 3 | 0.97 | 18.8 | 9.5 | +9.2 | 3 False+ |
| | With | 6 | 1.44 | 16.2 | 12.4 | +3.8 | Positive |
| | thrombosis | | (0.58-3.65) | (9.5-31.7) | (7.1-26.2) | (+1.3 to +6.7) | Positive |

Thus, 95% of the patients avoid imaging on the basis of a negative level of D-dimers adjusted as a function of the D-dimers generated equally by hypercoagulation (H), inflammation (I) and hyperfibrinolysis (F).

Among the false positives, 3 are at the threshold limit, 4 are cancers and 3 have a coagulation activation cause and can avoid imaging on the basis of a considerable shortening of the fibrin formation time, without generating false negatives, as shown in example 7.

### Example 7: Adjustment of D-Dimers by the Method of the Invention Compared with the Ratio [Ddi/Fibrinogen by the Clauss Method]

The protocol used in this example is protocol A. Step h) of the method used in this example is that of the first version of the method.

The adjustment of the D-dimers as a function of the D-dimers generated by hypercoagulation (H), inflammation (I) and hyperfibrinolysis (F) according to the first version of the method of the invention (step h)) is more effective than the ratio [D-dimers/fibrinogen], as shown by FIG. 12 and table 6 below. The patients with thrombosis are discriminated from those without thrombosis on the basis of a high level of adjusted D-dimers, which are multiplied by a factor of 5.3, as shown in table 6 below. Thus, 92% of the patients with a falsely positive level of D-dimers, that is to say 97% of the patients without thrombosis, are rendered negative with the adjustment of D-dimers according to step h), as shown by FIG. 12. Among the 7 false +, 4 are cancers and 3 are at the threshold limit.

TABLE 6
Adjustment of D-dimers by the optical method of the invention, compared with adjustment
by the ration [D-dimers/fibrinogen by the Clauss method], in the plasmas from patients
with a suspicion of thrombosis (with or without thrombosis, and with or without cancer)
(H: hypercoagulation, I: inflammation, F: hyperfibrinolysis, Ddi: D-dimers, Fib: fibrinogen)

| | Diagnosis | | Adjusted Ddi | | | Ratio | | |
|---|---|---|---|---|---|---|---|---|
| Plasmas | Ddi and/or | | [Ddi/(H + I + F)] | | | [Ddi/Fib] Clauss | | |
| from | imaging | N | Mean | Range | Result | Mean | Range | Result |
| Patients with | Ddi negative | 127 | 0.09 | 0.04-0.21 | Neg | 0.09 | 0.04-0.16 | Neg |
| suspicion of | Ddi positive | 88 | 0.31 | 0.11-1.56 | 7 False+ | 0.40 | 0.09-3.60 | 17 False+ |
| PE and/or | Without | | | | | | | |
| DVT with or | thrombosis | | | | | | | |
| without | Ddi positive | 21 | 1.65 | 0.52-10.5 | 0 False− | 1.35 | 0.38-7.71 | 2 False− |
| cancer | With | | | | | | | |
| | thrombosis | | | | | | | |
| Variation +/− thrombosis | | | ×5.3 | | | ×3.4 | | |
| Patients with | Without | 16 | 0.45 | 0.12-1.56 | 4 False+ | 0.75 | 0.09-3.60 | 7 False+ |
| suspicion of | thrombosis | | | | | | | |
| thrombosis | With | 6 | 1.44 | 0.58-3.65 | 0 False− | 1.18 | 0.38-3.0 | 1 False− |
| with cancer | thrombosis | | | | | | | |

| | | |
|---|---|---|
| Variation +/− thrombosis | ×3.2 | ×1.6 |

The false negatives with the ratio [D-dimers/fibrinogen] are detected by the method of the invention, which makes it possible not to miss a thrombosis.

There are fewer false positives by the method of the invention than with the ratio [D-dimers/fibrinogen], which makes it possible to reduce the number of patients diagnosed by imaging.

### Example 8: Influence of the Level of D-Dimers Adjusted According to the Method of the Invention, with Regard to the Probability of PE and/or of DVT

The protocol used in this step is protocol A. Step h) of the method used in this example is that of the first version of the method.

The threshold of the D-dimers adjusted according to the method of the invention was advantageously not adjusted as a function of age. This is because the level of D-dimers, the coagulation activation and the inflammation gradually increase as a function of age after the age of 50. As a result, the ratio is not adjusted as a function of age.

The higher the levels of D-dimers adjusted according to the method of the invention, the higher the denominator of the formula with the fibrin formation time and of the ratio with the DOD, the higher the probability of PE or of DVT.

The greater the extent of the PE and the more proximal rather than distal or superficial the DVT, the higher the adjusted D-dimers, as shown in table 7 below.

TABLE 7
Probability of PE and of DVT as a function of the level of D-dimers that
have been adjusted for hypercoagulation (H), inflammation (I) and hyperfibrinolysis
(F), (FFT: fibrin formation time, T: mean Control Time − 1 standard
deviation = 120 sec), FgDPs: fibrinogen degradation products, Ddi:
D-dimers, Act: coagulation activation state).

| | | | | | Probability | |
|---|---|---|---|---|---|---|
| | | Adjusted Ddi | Delta | FFT ≤ | PE and/or | Plasmas from patients |
| Number | Ddi | [Ddi/C + I + F] | FgDPs | T | DVT | with PE and/or DVT |
| 1 | 1.56 | 0.58 | / | 190 | Medium | Subsegmental PE |
| 3 | 3.60 | 0.92 | +2.6 | 157 | High | Segmental PE |
| 1 | 7.0 | 1.75 | +6.7 | 212 | High | Bilateral pulmonary |
| | | | | | | artery PE |
| 1 | 10.9 | 3.65 | +5.5 | 188 | High | Proximal and distal PE |
| 1 | 30.0 | 10.47 | +14.6 | 182 | Very high | Massive PE |
| 1 | 0.72 | 0.25 | / | 88 | Activation | Peroneal vein |
| | | | | | | inflammation |
| 2 | 1.44 | 0.49 | / | 162 | Low | Superficial DVT |
| 1 | 2.28 | 0.58 | +1.2 | 162 | Medium | Distal DVT |
| 1 | 3.60 | 0.91 | +2.7 | 218 | High | Proximal DVT |
| 1 | 7.70 | 1.62 | +9.3 | 192 | High | |
| 1 | 12.1 | 4.19 | +5.3 | 144 | High | Large jugular DVT |

### Example 9: Inter-Instrument Reproducibility of the Fibrin Formation Measurement

The protocol used in this example is protocol C on STA-R®, which was used as follows, according to steps a), b), c) and d) of the first or of the second version of the method.

The following are simultaneously added, by the instrument, to 8 cuvettes of the STA-R® Evolution Expert Series automated device (Stago):
200 μl of control-plasma sample: normal (STA® CCN); hypercoagulant (plasma depleted of protein S, STA DEF PS) and of hypofibrinolytic control plasma (plasma containing plasminogen activator inhibitor PAI-I, PAI 4C),
50 μl of a mixture of tissue factor and of phospholipids [TF 2 pM final+PL 4 μM final].

After stirring, and incubation for 300 seconds at 37° C., the automated device adds 50 μl of CaCl₂ at a final concentration of 16.7 mM, and stirs by means of the needle. Finally, the automated device measures the fibrin formation time and the variation in optical density DOD at the time to reach the plateau (TA); at the wavelength of 540 nm, as a function of time for 10 minutes.

The inter-instrument reproducibility of the method was determined on three STAR® instruments, as follows: 12 series of 2 measurements, that is to say 24 measurements, were carried out on each of the 3 instruments, for 5 consecutive days, using 2 bottles of each of the normal control plasmas (CCN) and pathological control plasmas (DPS:

control plasma depleted of protein S, hypercoagulant, PAI: hypofibrinolytic control plasma), that is to say 48 measurements for each control plasma. The aberrant values were eliminated according to Rosner.

The coefficients of variation (CV) obtained on the fibrin formation time FFT are all less than 6%; those obtained on the OD at the time of the plateau and on the maximum OD are all less than 3%, whatever the control plasma, as shown in table 8 below. The inter-STAR® variations are less than 2.5% whatever the parameter and the control plasma.

TABLE 8
Inter-instrument reproducibility of the measurement of fibrin formation
(CCN: normal control plasma, DPS: control plasma depleted of protein
S, hypercoagulant, PAI: hypofibrinolytic control plasma).

| N = 48 measurements | | | | | | | | |
|---|---|---|---|---|---|---|---|---|
| Fibrin formation time FFT | | | OD at the time of the | | | | | |
| (sec) | | | plateau | | | Maximum OD | | |
| Control plasma | | | | | | | | |
| CCN | DPS | PAI | CCN | DPS | PAI | CCN | DPS | PAI |

STAR ® 1

| | | | | | | | | | |
|---|---|---|---|---|---|---|---|---|---|
| mean | 100.4 | 82.4 | 117.8 | 2.200 | 1.977 | 2.280 | 2.223 | 2.004 | 2.306 |
| min | 86.0 | 78.0 | 102 | 2.157 | 1.751 | 2.217 | 2.174 | 1.768 | 2.239 |
| max | 110.0 | 86.0 | 126 | 2.278 | 2.053 | 2.347 | 2.310 | 2.071 | 2.384 |
| CV | 5.8% | 2.8% | 4.5% | 1.4% | 2.8% | 1.4% | 1.5% | 2.9% | 1.5% |

STAR ® 2

| | | | | | | | | | |
|---|---|---|---|---|---|---|---|---|---|
| mean | 99.7 | 83.1 | 118.2 | 2.222 | 2.015 | 2.309 | 2.337 | 2.043 | 2.337 |
| min | 88.0 | 78.0 | 100.0 | 2.073 | 1.788 | 2.248 | 2.265 | 1.804 | 2.265 |
| max | 116.0 | 90.0 | 138.0 | 2.280 | 2.114 | 2.381 | 2.422 | 2.154 | 2.422 |
| CV | 5.6% | 3.3% | 5.7% | 1.9% | 2.7% | 1.3% | 1.5% | 2.8% | 1.5% |

STAR ® 3

| | | | | | | | | | |
|---|---|---|---|---|---|---|---|---|---|
| mean | 100.1 | 84.2 | 118.7 | 2.245 | 2.030 | 2.328 | 2.265 | 2.057 | 2.352 |
| min | 90.0 | 76.0 | 106.0 | 2.192 | 1.879 | 2.261 | 2.213 | 1.904 | 2.279 |
| max | 110.0 | 90.0 | 134.0 | 2.323 | 2.112 | 2.374 | 2.365 | 2.129 | 2.395 |
| CV | 4.4% | 3.5% | 4.6% | 1.2% | 2.6% | 1.2% | 1.3% | 2.4% | 1.1% |

Inter-STAR ® variations

| | | | | | | | | | |
|---|---|---|---|---|---|---|---|---|---|
| mean | 99.9 | 83.2 | 118.2 | 2.222 | 2.007 | 2.306 | 2.275 | 2.035 | 2.332 |
| standard | 0.35 | 0.90 | 0.42 | 0.02 | 0.03 | 0.02 | 0.06 | 0.03 | 0.02 |
| deviation | | | | | | | | | |
| CV | 0.3% | 1.1% | 0.4% | 1.0% | 1.4% | 1.1% | 2.5% | 1.3% | 1.0% |

### Example 10: Repeatability of the Fibrin Formation Measurement

The protocol used in this example is protocol A on STA-R®, which was used as follows, according to steps a), b), c) and d) of the first or of the second version of the method.

The following are simultaneously added, by the instrument, to 8 cuvettes of the STA-R® Evolution Expert Series automated device (Stago):
200 μl of sample of citrated plasma from normal patients, or from patients with or without a suspicion of venous thrombosis,
50 μl of a mixture of tissue factor and of phospholipids [TF 2 pM final+PL 4 μM final].

After stirring, and incubation for 300 seconds at 37° C., the automated device adds 50 μl of CaCl₂ at 16.7 mM final concentration, and stirs by means of the needle. Finally, the automated device measures the fibrin formation time and the variation in optical density DOD at the time to reach the plateau (TA); at the wavelength of 540 nm, as a function of time, for 10 minutes.

The repeatability of the measurement duplicates was determined on 4 STAR® instruments, for each parameter of the method, and for each of the samples. The 154 plasmas from normal patients were tested with a first batch of reagents on one of the STAR® instruments, the 83 samples from patients with a suspicion of thrombosis were tested with a second batch of reagent on the other 3 STAR® instruments for the D-dimers, the fibrin formation time, the DOD at the plateau and the fibrinogen.

The mean and the standard deviation of the variations obtained on each of the parameters are reported in tables 9 and 10 below.

The mean of the variations obtained on the fibrin formation measurements is less than 2% for the 154 normal patients; it is less than 1% for the 83 patients with and without venous thrombosis, as shown in tables 9 and 10.

The measurements of the parameters of fibrin formation are as repeatable as those of fibrinogen in the Clauss method.

The measurements of the specific D-dimers are as repeatable as those of the D-dimers and of the ratio [D-dimers/fibrinogen]; the means of the variations is less than 2.3%.

Among the 5 samples rendered positive by specific D-dimers and negative by the ratio [Ddi/Fib] on the duplicates, 2 are from patients with a pulmonary embolism confirmed in imaging, 2 have coagulation hyperactivation and 1 is from a patient with a cardiopathy.

Thus, 2 patients with a PE, found to be falsely negative by the ratio [Ddi/Fib], can be diagnosed by the method of the invention; while a single patient without PE requires imaging by the method of the invention.

TABLE 9
Repeatability of the duplicates of fibrin formation
measurement in the samples from normal patients.

| | | Fibrin formation | | | |
|---|---|---|---|---|---|
| | | measurements | | Measurement variations | |
| Samples from normal patients | | | Standard | | Standard |
| Number of duplicates | | Mean | deviation | Mean | deviation |
| Fibrin formation time FFT | 150 | 134 | 24 | −1.1% | 7.1% |
| (seconds) | | 82-206 | | | |
| DOD at the plateau | 150 | 0.937 | 0.20 | −0.5% | 1.8% |
| (540 nm) | | 0.618-1.423 | | | |

TABLE 10
Repeatability of the duplicates of measurement of fibrin formation, of D-dimers and D-
dimers adjusted according to the method of the invention, and also of the ratio [D-
dimers/fibrinogen] in the samples from patients with and without thrombosis in imaging.

| Samples from patients with and | | Fibrin formation measurements | | | Measurement variations | |
|---|---|---|---|---|---|---|
| without thrombosis in imaging | | | | Standard | | Standard |
| Number of duplicates | | Mean | | deviation | Mean | deviation |
| Fibrinogen (g/l) | 83 | 3.97 | 1.92-8.40 | 1.10 | 0.4% | 3.0% |
| Fibrin formation time FFT | 83 | 151.6 | 88-382 | 41.4 | 0.9% | 5.0% |
| (seconds) | | | | | | |
| DOD at the plateau (540 nm) | 83 | 1.262 | 0.559-2.241 | 335 | 0.2% | 2.0% |
| Positive D-dimers >0.5 | 30 | 2.20 | 0.52-12.13 | 3.15 | 2.1% | 7% |
| μg/ml | | | | | | |
| Ratio [D-dimers/fibrinogen] | 30 | 0.67 | 0.12-4.92 | 1.14 | 2.1% | 6.7% |
| Negative ratio | 25 | 0.28 | 0.12-0.53 | 0.13 | 2.4% | 7.3% |
| Positive ratio | 5 | 2.61 | 0.60-4.92 | 1.85 | 0.2% | 1.8% |
| D-dimers specific for | 30 | 0.71 | 0.14-4.28 | 1.00 | 2.3% | 6.8% |
| thrombosis (μg/ml) | | | | | | |
| Negative specific Ddi | 21 | 0.30 | 0.14-0.50 | 0.11 | 3.6% | 7.7% |
| Positive specific Ddi | 9 | 1.66 | 0.51-4.28 | 1.44 | −0.6% | 1.8% |

### Example 11: Adjustment of D-Dimers According to the Method of the Invention with Another Reagent for Assaying D-Dimers

The protocol used in this example is protocol A. Steps e) to h) of the method used in this example are those of the first version of the method.

The D-dimers are adjusted as a function of the D-dimers generated by hypercoagulation (H) by the formula of example 2, of the inflammation (I) by the logarithmic function of example 4 and the ratio R of example 5, and of the hyperfibrinolysis (F) by the linear equations of example 6, with the Instrumentation Laboratory (IL) method for assaying D-dimers.

The adjustment of the D-dimers as a function of the D-dimers generated by hypercoagulation makes it possible to subtract the D-dimers generated by hypercoagulation in coagulation activation states, but does not make it possible to discriminate the patients with and without thrombosis, as shown in table 11.

TABLE 11
Adjustment of the D-dimers with the IL method for assaying D-
dimers as a function of the D-dimers generated by hypercoagulation
in plasmas from patients with a suspicion of thrombosis.

| Plasmas | Diagnosis | | Level of initial D- | | Level of adjusted D- | |
|---|---|---|---|---|---|---|
| from | D-dimers | | dimers (μg/ml) | | dimers/hypercoag (μg/ml) | |
| patients | and/or imaging | Number | Mean | Range | Mean | Range |
| With a | Ddi negative | 120 | 0.43 | 0.11-3.32 | 0.46 | 0.09-3.35 |
| suspicion of | Ddi positive | 79 | 1.75 | 0.20-7.34 | 2.12 | 0.21-16.7 |
| thrombosis | without | | | | | |
| | thrombosis | | | | | |
| | Ddi positive | 15 | 8.42 | 1.39-25.4 | 10.9 | 1.82-34.7 |
| | with | | | | | |
| | thrombosis | | | | | |

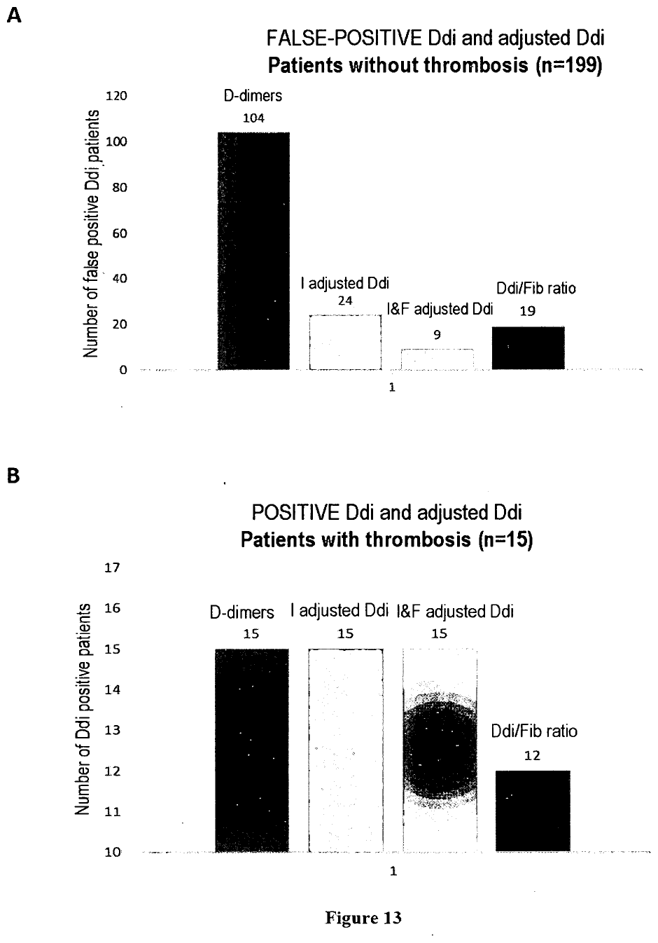

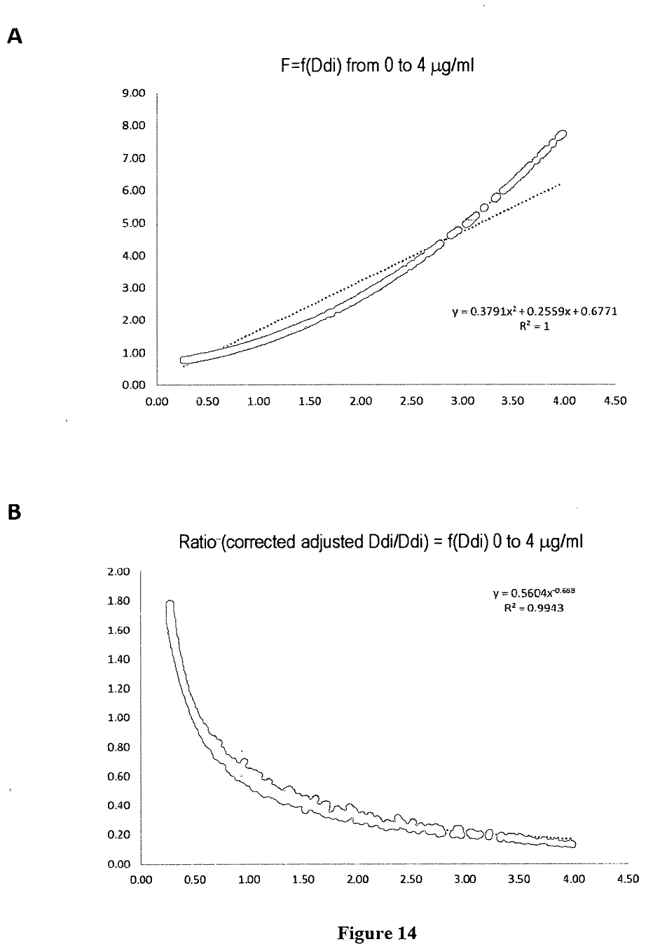

The adjustment of the D-dimers as a function of the D-dimers generated by inflammation makes it possible to discriminate the patients with and without thrombosis, as shown in FIG. 13 and table 12. Thus, approximately 90% of the patients can avoid imaging with one or other of the 2 Stago and IL reagents, on a negative adjusted D-dimer level.

TABLE 12
Adjustment of the D-dimers with the IL method for assaying D-dimers
as a function of the D-dimers generated by hypercoagulation and by
inflammation in plasmas from patients with a suspicion of thrombosis.

| | | | Level of adjusted | | Level of adjusted | | |
|---|---|---|---|---|---|---|---|
| Plasmas | Diagnosis | | D-dimers (μg/ml)/ | | D-dimers (μg/ml)/ | | Result |
| from | Ddi and/or | | hypercoag H | | inflammation I | | False+ |
| patients | imaging | N | Mean | Range | Mean | Range | False− |
| Plasmas from | Ddi negative | 120 | 0.46 | 0.09-3.35 | 0.12 | 0.02-1.04 | 2 F+ |
| patients | Ddi positive | 79 | 2.12 | 0.21-16.7 | 0.45 | 0.06-2.47 | 22 F+ |
| with a | without | | | | | | |
| suspicion of | thrombosis | | | | | | |
| thrombosis | Ddi positive | 15 | 10.9 | 1.82-34.7 | 2.45 | 0.49-8.84 | 1 lim |
| | with | | | | | | |
| | thrombosis | | | | | | |

The adjustment of the D-dimers as a function of the D-dimers generated by hyperfibrinolysis (F), in addition to hypercoagulation (H) and inflammation (I), makes it possible to discriminate more patients, as shown in FIG. 13 and tables 13 and 14 below. Thus, more than 95% of the patients can avoid imaging, on the basis of a negative level of D-dimers adjusted according to the method of the invention. Among the 9 false +, 3 are cancers with metastases, 3 are at the threshold limit (≤0.60) and 3 are elderly patients (>80 years old).

TABLE 13
Discrimination of the plasmas from patients with a suspicion of thrombosis
(with or without thrombosis), by the method of the invention, with
the IL method for assaying D-dimers, as a function of the D-dimers
generated by hyperfibrinolysis with deduction of the FgDPs.

| Plasmas | Diagnosis | | | | FgDP | FgDP | |
|---|---|---|---|---|---|---|---|
| from | Ddi positive | | Adjusted | FgDP | Adjusted | Threshold ≥ | |
| patients | and imaging | N | Ddi | Ddi | Ddi | 1.0 | Result |
| With | Ddi negative | 120 | 0.12 | / | / | / | Neg |
| suspicion of | Without | 79 | 0.45 | 5.67 | 5.52 | / | 11 F+ |
| thrombosis | thrombosis | | | | | | |
| | With | 15 | 2.45 | 23.4 | 17.7 | +5.66 | Positives |
| | thrombosis | | | | | | 1 limit |

This adjustment according to the method of the invention is more effective than the ratio [D-dimers/fibrinogen], as shown in FIG. 13 and table 14 below. The false negatives with the ratio [D-dimers/fibrinogen] are detected by the method of the invention, which makes it possible not to miss a thrombosis. There are fewer false positives by the method of the invention than with the ratio [D-dimers/fibrinogen], which makes it possible to reduce the number of patients diagnosed using imaging.

TABLE 14
Comparison of the adjustment of D-dimers by the optical method of the invention
with the IL reagent for assaying D-dimers and by the ratio [D-dimers/fibrinogen],
in the plasmas from patients with or without thrombosis (H: hypercoagulation,
I: inflammation, F: hyperfibrinolysis, Ddi: D-dimers, Fib: fibrinogen).

| Plasmas | Diagnosis | | Adjusted Ddi | | | Ratio [Ddi/Fib] | | Result |
|---|---|---|---|---|---|---|---|---|
| from | Ddi and/or | | [Ddi/(C+ I + F)] | | | (Clauss) | | False+ |
| patients | imaging | Number | Mean | Range | Result | Mean | Range | False− |
| With a | Ddi negative | 120 | 0.12 | 0.02-0.41 | 0 F+ | 0.12 | 0.01-1.01 | 2 F+ |
| suspicion of | Ddi positive | 79 | 0.37 | 0.06-2.23 | 9 F+ | 0.41 | 0.05-2.26 | 17F+ |
| PE and/or | Without | | | | | | | |
| DVT | thrombosis | | | | | | | |
| | Ddi positive | 15 | 2.45 | 0.49-8.84 | 1 lim | 2.01 | 0.37-6.52 | 3 F− |
| | With | | | | | | | |
| | thrombosis | | | | | | | |

### Example 12: Diagnostic Performance Levels of the Method According to the Invention for Patients with a Suspicion of Thrombosis (with or without Thrombosis, with or without Cancer) as a Function of Age

The protocol used in this example is protocol A. Steps e) to h) of the method used in this example are those of the second version of the method.

The level of D-dimers which result from intravascular fibrin degradation, R, was calculated according to step e′) by adjusting the level of D-dimers of the sample as a function of the level of D-dimers generated by inflammation, and by correcting this adjusted level for the low levels of D-dimers (<4 μg/ml). The level of D-dimers generated by hyperfibrinolysis was calculated according to step g′), for each of the samples classified as a function of inflammation. The ratio TA/FFT was then compared with respect to a threshold, in order to exclude or to diagnose a thrombosis in the patient, or to turn attention to a coagulation activation state in the patient.

A total of 218 patients with a suspicion of thrombosis (with or without thrombosis, with or without cancer) were tested. Among these 218 patients tested, 47% were more than 50 years old, 17% were more than 75 years old and 15% had a cancer. The diagnosis of PE and/or of DVT was excluded on the basis of an imaging test or of a negative assay of D-dimers according to clinical probability, in 199 patients (91.3%). The diagnosis of PE and/or of DVT was confirmed on the basis of a positive imaging test in 19 patients, that is to say 8.7%. The exclusion and/or the diagnosis of a thrombosis or of a coagulation activation state was carried out for the 218 patients using the second version of the method of the invention. The diagnostic performance levels of the method were compared to those of the usual assaying of D-dimers and to those of the age-adjusted D-dimers.

The diagnostic performance levels of the method of the invention were then determined on a larger number of patients with a suspicion of thrombosis (with or without thrombosis, with or without cancer). On a total of 796 patients tested, 49% were more than 50 years old, 19% were more than 75 years old and 7.3% had a cancer. The diagnosis of PE and/or of DVT was excluded in 730 patients (91.7%). The diagnosis of PE and/or of DVT was confirmed in 66 patients, that is to say 8.3%.

The clinical usefulness of the determination of the level of D-dimers is limited in elderly patients. This is because the threshold of D-dimers must be adjusted with age in order to exclude pulmonary embolism (Righini et al., JAMA, 2014, 311: 1117-1124). This adjusted threshold corresponds to the age multiplied by 10 for patients 50 years old and older. The combination of the D-dimer threshold adjusted for age and of the pretest evaluation of the clinical probability is combined with a large number of patients in whom pulmonary embolism might have been excluded (Righini et al., JAMA, 2014, 311: 1117-1124). Methods for pretest evaluation of the clinical probability of pulmonary embolism (PE) and of deep vein thrombosis (DVT) are known in the art (see Kolok et al., Arch. Intern. Med., 2008, 168: 21-31, and Wells et al., NEJM, 2003, 349: 1227-1235, respectively).

The threshold of the D-dimers adjusted according to the method of the invention was advantageously not adjusted as a function of age. This is because the levels of D-dimers, the coagulation activation and the inflammation gradually increase as a function of age after the age of 50. As a result, the ratio is not adjusted as a function of age. The higher the levels of D-dimers adjusted according to the method of the invention, the higher the denominator of the ratio with the DOD, the higher the probability of PE or of DVT.

For the 218 patients with a suspicion of thrombosis, the method of the invention makes it possible to further exclude more patients (approximately 20% more) than the usual assaying of D-dimers or of age-adjusted D-dimers, as shown in table 15 below. Thus, more than 75% of the patients, tested by the method of the invention, do not need supplementary imaging tests.

The performance levels in table 15 are given at the usual threshold of 0.50 μg/ml for the D-dimers and the age-adjusted D-dimers, and at the threshold of 0.51 μg/ml in this example with the method of the invention. The sensitivity of the three methods is 100% given that there was no falsely negative diagnosis; on the other hand, the specificity of the method of the invention is much higher than that of the other two methods, as shown in table 1.5. The positive likelihood ratio is higher than that of the other two methods, confirming that the patient has three times more chance of having a thrombosis when the result is positive, than with the other methods.

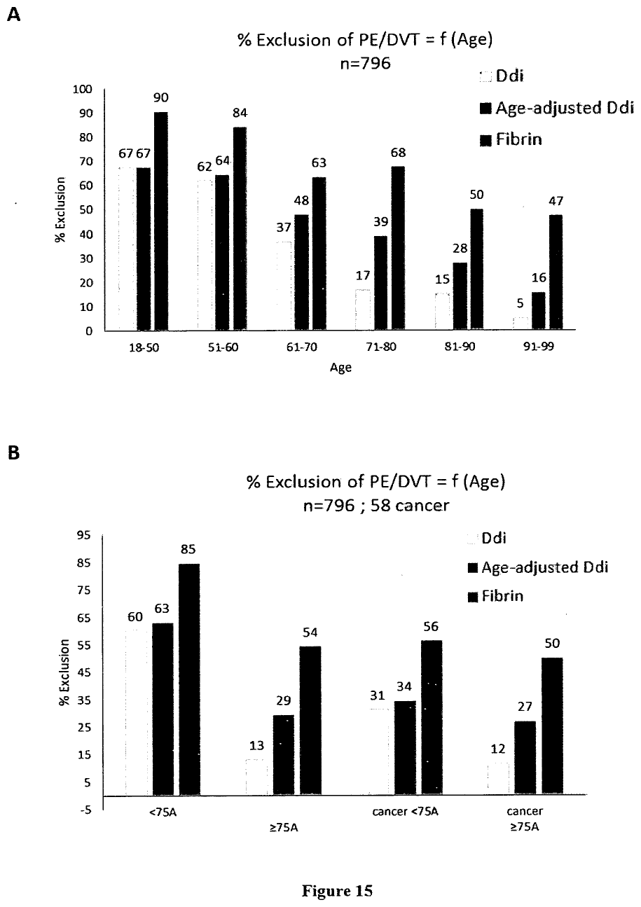

Similar diagnostic performance levels are found when the method according to the invention is applied to a larger number of patients. Among the 796 patients with a suspicion of thrombosis, the method makes it possible to exclude 79% of patients (more than 20% more than the other methods) as shown in table 16, and regardless of age, as shown in table 17 and FIG. 15(A). The method according to the invention also makes it possible to exclude more patients (<75 years of age) suffering from a cancer (see FIG. 15(B)). Finally, the method according to the invention allows a better exclusion not only of elderly patients and patients suffering from a cancer, but also of elderly patients suffering from a cancer (see FIG. 15(B)). The results on a larger number of patients demonstrate a greater specificity of the method according to the invention compared with the reference method (see table 16). The positive predictive value is approximately two times higher than that of the reference method.

TABLE 15
Comparison of the diagnostic performance levels of the method of the invention with
those of the assays of D-dimers and of age-adjusted D-dimers, for the 218 patients
with a suspicion of thrombosis (19 with thrombosis and 199 without thrombosis).

| | Diagnostic performance levels N = 218 patients | | | | |
|---|---|---|---|---|---|
| | Number of | | | | |
| | patients | | | | |
| | excluded for the | | | Positive | Negative |
| | diagnosis of PE | Sensitivity | Specificity | likelihood ratio | likelihood ratio |
| | or of DVT | % | % | (LR+) | (LR−) |
| Method of | 166/218 | 100% | 83.2% | 6.03 | 0.00 |
| the invention | (76.2%) | | | | |
| D-dimers | 115/218 | 100% | 57.8% | 2.37 | 0.00 |
| | (52.8%) | | | | |
| Age-adjusted | 123/218 | 100% | 61.8% | 2.62 | 0.00 |
| D-dimers | (56.4%) | | | | |

TABLE 16
Comparison of the diagnostic performance levels of the method
of the invention with those of the assays of D-dimers and of
age-adjusted D-dimers, for the 796 patients with a suspicion
of thrombosis (66 with thrombosis and 730 without thrombosis).

| | Diagnostic performance levels N = 796 patients | | | |
|---|---|---|---|---|
| | % | | Speci- | PPV (positive |
| | Exclusion | Sensitivity | ficity | predictive value) |
| Method of the | 79% (+28%) | 99.8% | 86% | 39% |
| invention | | | | (X2) |
| D-dimers | 51% | 100% | 56% | 17% |
| Age-adjusted | 57% (+6%) | 100% | 62% | 17% |
| D-dimers | | | | |

TABLE 17
Level of D-dimers (D-di) resulting from intravascular fibrin
degradation according to the method of the invention, for the
386 patients under the age of 50 and the 379 patients over
the age of 50 in whom the level of D-dimers (D-di) is <5.1
μg/ml, among the 796 patients with a suspicion of thrombosis.

| | | | | Method of the | |
|---|---|---|---|---|---|
| | Diagnosis | | D-di | invention | |
| Age | PE and/or DVT | n | μg/ml | D-di μg/ml | Average age |
| <50A | Positive | 13 | 2.23 | 0.69 | 38 years old |
| N = 386 | PE or DVT | | | 0.52-1.62 | |
| | Negative | 375 | 0.55 | 0.51 | 35 years old |
| | | | | 0.45-1.39 | |
| >=50A | Positive | 36 | 2.25 | 0.65 | 72 years old |
| N = 379 | PE or DVT | | | 0.55-0.95 | |
| | Negative | 343 | 1.04 | 0.56 | 68 years old |
| | | | | 0.46-1.76 | |

The levels of D-dimers resulting from intravascular fibrin degradation according to the method of the invention are similar for the patients with a diagnosis of thrombosis regardless of their age (average age 38 or 72 years old). On the other hand, in the patients without thrombosis, the D-dimers are higher as a function of age, as expected.

### Example 13: Diagnostic Performance Levels of the Method According to the Invention by Patient Group

The protocol used in this example is protocol A. Steps e) and h) of the method used in this example are those of the second version of the method.

The level of D-dimers which result from intravascular fibrin degradation, R, was calculated according to step e′) by adjusting the level of D-dimers of the sample as a function of the level of D-dimers generated by inflammation, and by correcting this adjusting level for the low levels of D-dimers (<4 μgimp. The level of D-dimers generated by hyperfibrinolysis was calculated according to step g′), for each of the samples classified as a function of inflammation. The ratio TA/FFT was then compared with respect to a threshold, in order to exclude or to diagnose a thrombosis in the patient, or to direct attention to a coagulation activation state in the patient.

The diagnostic performance levels of the method of the invention were determined on a total of 796 patients with a suspicion of thrombosis using the second version of the method of the invention. The results obtained by means of the method of the invention on the 8.3% of patients with a thrombosis, the 7.3% of patients with a cancer, on the patients with a predisposition to thrombosis, those with a hypercoagulation state and those with a coagulation activation state are presented in tables 18 to 21 below.

### Diagnosis of Pulmonary Embolism (PE) and of Deep Vein Thrombosis (DVT)

The diagnostic performance levels of the method of the invention, obtained on 46 patients with a pulmonary embolism (PE), 16 patients with a deep vein thrombosis (DVT), 4 patients with both a PE and a DVT, 15 patients of whom have a history (HIST) of venous thrombosis, are described in table 18.

TABLE 18
Diagnostic performance levels of the method of the invention,
obtained on the 66 patients with a diagnosis of thrombosis
among the 796 patients with a suspicion of thrombosis.

| | | | | Method of the | |
|---|---|---|---|---|---|
| Diagnosis of | | Ddi | Inflammation | invention Ddi | True positive (TP) |
| thrombosis | n | (μg/ml) | (DOD) | (μg/ml) | performance levels |
| PE+ positive | 46 | 5.2 | 4.8 | 1.43 | 97.8% TP |
| | | 0.68-30 | 2.6-9.9 | 0.52-7.64 | 1 non-serious SSPE* |
| DVT+ positive | 16 | 4.1 | 4.4 | 1.28 | 100% TP |
| | | 0.77-13 | 2.6-6.6 | 0.55-5.05 | |
| PE+ DVT+ positive | 4 | 5.0 | 4.3 | 1.36 | 100% TP |
| | | 1.15-10 | 3.3-5.4 | 0.61-3.03 | |
| HIST PE or DVT | 15 | 5.8 | 4.5 | 1.83 | 100% TP |
| | | 0.68-20 | 2.6-8.4 | 0.54-7.24 | |
| Negative superficial | 3 | 1.1 | 4.0 | 0.60 | 2 false positive |
| VT | | | | | |
| Negative venous | 2 | 0.52 | 4.2 | 0.50 | 2 true negatives |
| insufficiency | | | | | |

*SSPE: subsegmental pulmonary embolism.

The results obtained show that the method according to the invention makes it possible to diagnose close to 100% of patients suffering from venous thromboembolism, including in the event of pulmonary infarction, the level of inflammation of which is high, and in the event of non-serious, isolated subsegmental pulmonary embolism, as shown in table 19. It should be noted that the levels of D-dimer which result from intravascular fibrin degradation that are determined are higher in the case of a history (HIST) of venous thromboembolism (pulmonary embolism or deep vein thrombosis), as shown in table 18.

TABLE 19
Diagnostic performance levels of the method of the invention,
obtained on the 66 patients with a diagnosis of thrombosis
among the 796 patients with a suspicion of thrombosis.

| | | | | Inflammation | Method of the invention |
|---|---|---|---|---|---|
| | | | | (DOD) | Ddi (μg/ml) |
| Diagnosis of thrombosis | N | D-dimers | | N: 2 to 4.5 g/l | TP = True positives |
| Massive, bilateral or | 24 | 7.1 | 1.2-30.0 | 4.8 | 1.91 |
| segmental PE | | | | 2.8-8.4 | 0.57-7.52 (100% TP) |
| Distal PE | 7 | 1.7 | 0.89-2.77 | 5.4 | 0.60 |
| | | | | 3.7-9.9 | 0.55-0.65 (100% TP) |
| Pulmonary infarction | 7 | 2.5 | 1.1-5.1 | 5.8 | 0.73 |
| | | | | 3.1-8.4 | 0.52-1.62 (100% TP) |
| SSPE associated with PE | 6 | 3.3 | 1.15-5.1 | 5.0 | 0.80 |
| | | | | 3.1-7.5 | 0.52-1.62 (100% TP) |
| Isolated SSPE | 6 | 1.9 | 0.68-3.8 | 4.2 | 0.62 |
| | | | | 2.6-6.9 | 0.54-0.58 (1 False negative) |

### Thrombosis Predisposition States

The 113 patients presenting a predisposition to thrombosis who were tested in this study were patients suffering from a known cancer (58 patients, including 47 diagnosed negative for VTE, and 11 diagnosed positive), patients suffering from an unknown cancer (21 patients, including 17 diagnosed negative and 4 diagnosed positive), pregnant women (14 patients, all diagnosed negative), women having given birth (4 patients, all diagnosed negative), patients in post-operative phase (2 patients, all diagnosed negative), and patients who had just taken a lengthy trip (14 patients, all diagnosed negative, except 1 diagnosed positive).

TABLE 20
Diagnostic performance levels of the method of the invention, obtained on 113
patients with a thrombosis predisposition state, 16 of whom have a diagnosis
of thrombosis, among the 796 patients with a suspicion of thrombosis.

| Patients with a | | | | | Method of | |
|---|---|---|---|---|---|---|
| thrombosis | Diagnosis | | | | the | |
| predisposition | of | | | Inflammation | invention | Performance |
| state | thrombosis | n | Ddi | (DOD) | Ddi (μg/ml) | levels |
| Known cancer | Positive | 11 | 5.0 | 4.7 | 1.50 | 100% true |
| (n = 58) | | | | | | positives |
| | Negative | 47 | 1.7 | 4.7 | 0.68 | 53% exclusion |
| Unknown | Positive | 4 | 6.4 | 4.2 | 2.0 | 100% true |
| cancer (n = 21) | | | | | | positives |
| | Negative | 17 | 2.0 | 4.8 | 0.67 | 48% exclusion |
| Pregnancy | Negative | 14 | 0.78 | 3.8 | 0.56 | 93% exclusion |
| Childbirth | Negative | 4 | 0.67 | 3.5 | 0.54 | 100% exclusion |
| Post-operative | Negative | 2 | 1.40 | 3.9 | 0.61 | 100% exclusion |
| Trip | Positive | 1 | 2.23 | 8.4 | 0.58 | 100% true |
| N = 14 | | | | | | positives |
| | Negative | 13 | 0.76 | 3.7 | 0.54 | 93% exclusion |

The results obtained in the patients with a thrombosis predisposition state clearly show that the method according to the invention makes it possible to diagnose 100% of the patients suffering from venous thromboembolism. The method of the invention also allows the exclusion of more than 93% of the non-cancer patients, as shown in table 20. In the patients suffering from cancer, the method of the invention allows the exclusion of 50% of the patients, compared with 22% with the usual method for determining D-dimers.

### Hypercoagulation State

The 436 patients with a hypercoagulation state who were tested in this study were patients suffering from thrombophilia (4 patients, all diagnosed negative for VTE, except 1 diagnosed positive), patients suffering from renal insufficiency (26 patients, all diagnosed negative, except 1 diagnosed positive), patients having suffered a trauma, a fall or a fracture (13 patients, all diagnosed negative), and a group of subjects aged 50 or over (393 subjects, including 357 diagnosed negative and 36 diagnosed positive).

TABLE 21
Diagnostic performance levels of the method of the invention, obtained
on 436 patients with a hypercoagulation state, 38 of whom have a diagnosis
of thrombosis, among 796 patients with a suspicion of thrombosis.

| Patients with a | | | | | Method of | |
|---|---|---|---|---|---|---|
| hyper- | Diagnosis | | | | the | |
| coagulation | of | | | Inflammation | invention | Performance |
| state | thrombosis | n | Ddi | (DOD) | Ddi (μg/ml) | levels |
| Thrombophilia | Positive | 1 | 4.0 | 4.8 | 0.82 | True positive |
| | Negative | 3 | 0.37 | 3.8 | 0.48 | 100% exclusion |
| Renal | Positive | 1 | 2.2 | 8.4 | 0.58 | True positive |
| insufficiency | Negative | 25 | 3.0 | 5.1 | 0.78 | 50% exclusion |
| (RI) | | | | | | |
| nephropathy | | | | | | |
| Trauma, fall, | Negative | 13 | 1.86 | 3.9 | 0.70 | 77% exclusion |
| fractures | | | | | | |
| Age ≥ 50 | Positive | 36 | 2.25 | 5.0 | 0.65 | 100% true |
| Average age: | | | | | | positives |
| 69 | Negative | 357 | 1.21 | 4.4 | 0.58 | 77% exclusion |
| | | | | | | compared with |
| | | | | | | 94% for < 50 |
| | | | | | | years old |

The results obtained in the patients with a hypercoagulability state clearly show that the method according to the invention makes it possible to diagnose 100% of the patients suffering from venous thromboembolism. The method of the invention also allows the exclusion of more than 75% of the patients, including in the elderly patients, as shown in table 21. Among the elderly patients not excluded by the method of the invention, ⅓ have a cancer and ⅔ are suffering from renal insufficiency or from coagulation activation.

### Coagulation Activation States

The 144 patients with a coagulation activation state who were tested in this study were patients suffering from infections or from sepsis (5 patients, all diagnosed negative for VTE), patients suffering from pneumopathy, bronchitis or respiratory insufficiency (71 patients, all diagnosed negative, except 1 diagnosed positive), patients suffering from inflammatory diseases (6 patients, all diagnosed negative), patients suffering from gastritis (7 patients, all diagnosed negative), patients suffering from cardiomyopathies (45 patients, all diagnosed negative, except 1 diagnosed positive), and patients with a history of stroke (10 patients, all diagnosed negative).

TABLE 22
Diagnostic performance levels of the method according to the invention, obtained
on 144 patients with a coagulation activation state, 2 of whom have a diagnosis
of thrombosis, among the 796 patients with a suspicion of thrombosis.

| Patients with a | | | | | Method of | |
|---|---|---|---|---|---|---|
| hyper- | Diagnosis | | | | the | |
| coagulation | of | | | Inflammation | invention | Performance |
| state | thrombosis | n | Ddi | (DOD) | Ddi (μg/ml) | levels |
| Infections, | Negative | 5 | 2.88 | 5.3 | 0.80 | 100% exclusion |
| sepsis | | | | | | |
| Pneumopathy, | Positive | 1 | 2.76 | 9.9 | 0.56 | True positive |
| bronchitis, | Negative | 70 | 0.86 | 5.0 | 0.53 | 83% exclusion |
| respiratory | | | | | | |
| insufficiency | | | | | | |
| Inflammatory | Negative | 6 | 2.16 | 4.3 | 0.85 | 100% exclusion |
| diseases | | | | | | |
| Gastritis | Negative | 7 | 0.72 | 4.3 | 0.51 | 86% exclusion |
| Cardio- | Positive | 1 | 2.77 | 4.1 | 0.65 | True positive |
| myopathies | Negative | 44 | 1.19 | 4.3 | 0.57 | 66% exclusion |
| (SCA, heart | | | | | | |
| failure, etc.) | | | | | | |
| HIST stroke | Negative | 10 | 0.74 | 4.0 | 0.53 | 90% exclusion |

The results obtained in the 144 patients with a coagulation activation state clearly show that the method according to the invention makes it possible to diagnose 100% of the patients suffering from venous thromboembolism and also to exclude 80% of the negative patients (with the exception of the elderly patients who have a cardiomyopathy, of average age: 80), as shown in table 22.

1. A method for assaying D-dimers specific for venous thromboembolism in a blood sample from a patient, said method comprising, on the one hand, the assaying of D-dimers in the sample in order to obtain the level of D-dimers in the sample (Ddi_{S}), and on the other hand, the dynamic measurement of fibrin formation in this same sample, said dynamic measurement comprising steps of:
a) initiating the activation of coagulation in the sample without triggering it;
b) incubating the mixture obtained in step a), and triggering, in the incubated sample, the generation of thrombin and the formation of a fibrin clot;
c) measuring the time variation of at least one property of the sample obtained in b), in which the fibrin clot forms;
d) establishing the formation profile of the fibrin clot analyzed in c), and extracting from said profile the fibrin formation time (FFT) measured at the point of inflection of the tangent of the profile curve, the value of the property (Vp_{(TA)}) of the sample measured at the time to reach (TA) the fibrin polymerization plateau;
e) calculating the level of D-dimers resulting from intravascular fibrin degradation (R):
e1) by adjusting the level of D-dimers of the sample (Ddi_{S}) as a function of the level of D-dimers generated by hypercoagulation using FFT determined in d), in order to obtain the level of D-dimers adjusted as a function of the hypercoagulation (Ddi_{S/HC}); and
e2) by adjusting the level of adjusted D-dimers Ddi_{S/HC} obtained in e1) as a function of the level of D-dimers generated by inflammation using Vp(T_{A}) determined in d), in order to obtain R;
f) comparing the level of D-dimers resulting from the fibrin degradation R obtained in e), with respect to a threshold, preferably to the threshold of 0.5 μg/ml;
g) determining the level of fibrinogen degradation products generated by hyperfibrinolysis (FgDP_{(HF)}) present in the coagulation activation states, using the level of D-dimers resulting from the fibrin degradation, R, obtained in e) and the level of D-dimers of the sample (Ddi_{S});
h) comparing the FgDP_{(HF)} level obtained in g) with respect to a threshold, and comparing the fibrin formation time (FFT) obtained in d) with respect to a threshold.

2. The method according to claim 1, wherein, in step e), calculating the level R expressed in initial fibrinogen units (FEUs) is carried out:
e1) by calculating the level of D-dimers adjusted as a function of hypercoagulation (Ddi_{S/HC}) using the following equation:
$${Ddi}_{S / HC} = {Ddi}_{S} \times \frac{FFT}{Control Time}$$
wherein the Control Time is the average fibrin clot formation time of samples from healthy patients with no suspicion of thrombosis, measured according to steps a)-d), and
e2) by calculating the level R using the following equation:
$$R = \frac{{Ddi}_{S / HC}}{{[ Fib ]}_{( Vp ( TA ) )}}$$
wherein [Fib]_{(Vp(TA))} is the fibrinogen concentration deduced for the value of the property Vp_{(TA)} on a standard curve having the equation:
y=a ln(x)−b,
wherein:
y is the value of the property measured at the time to reach (TA) the fibrin polymerization plateau,
x is the fibrinogen concentration,
a and b are the constants of the logarithmic equation which links the level of the fibrin plateau, and the fibrinogen concentration,
the standard curve having been established for blood samples, the fibrinogen concentration of which has been determined and the value of the property (Vp_{(TA)}) of which has been determined by steps a)-d).

3. The method according to claim 1, wherein, in step f), a level R obtained in e), below the threshold, preferably below the threshold of 0.5 μg/ml, makes it possible to exclude a thrombosis in the patient, and a level R obtained in e), above the threshold, preferably above the threshold of 0.5 μg/ml is indicative of the possibility of a thrombosis in the patient.

4. The method according to claim 1, wherein step g) comprises steps of:
g1) determining the level of FgDP corresponding to the level of D-dimers in the sample (Ddi_{S}) on a standard curve established using blood samples with known levels of FgDP and known levels of D-dimers,
g2) determining the level of FgDP corresponding to the adjusted level of D-dimers (R) obtained in step e) on the standard curve used in g1), and
g3) determining the level of FgDP generated by hyperfibrinolysis (FgDP_{(HF)}) by subtracting the level of FgDP obtained in g2) from the level of FgDP obtained in g1).

5. The method according to claim 4, wherein step h) comprises steps of:
h1) comparing FgDP_{(HF)} to a threshold, in particular a threshold of 1 μg/ml, wherein FgDP_{(HF)} below the threshold or negative makes it possible to exclude thrombosis in the patient, and FgDP_{(HF)} above the threshold is indicative of a possibility of thrombosis in the patient,
h2) comparing FFT to a threshold, in particular to a threshold equal to [Control Time of e1)−1 standard deviation], for example a threshold of 120 seconds for a Control Time of 135 seconds, where FFT below the threshold is indicative of a patient without thrombosis but having an acute coagulation activation state, and a FFT above the threshold is indicative of a thrombosis in the patient.

6. The method according to claim 5, wherein:
when a thrombosis has been diagnosed in step h2), the level of D-dimers specific for venous thromboembolism (Ddi_{VTE}) is the level of D-dimers, R, obtained in step e),
when a thrombosis has been excluded in steps h1) and h2) but the level R obtained in step e) is above a threshold, preferably above the threshold of 0.5 μg/ml, the method also comprises a step consisting of calculating the level of D-dimers specific for venous thromboembolism (Ddi_{VTE}) using the following equation:
$${Ddi}_{VTE} = 0.5 \times \frac{R}{{Ddi}_{S}}$$
wherein Ddi_{S} is the level of D-dimers in the sample.

7. A method for assaying D-dimers specific for venous thromboembolism in a blood sample from patient, said method comprising, on the one hand, the assaying of D-dimers in the sample in order to obtain the level of D-dimers in the sample (Ddi_{S}), and on the other hand, the dynamic measurement of the fibrin formation of this same sample, said dynamic measurement comprising steps of:
a) initiating the activation of coagulation in the sample without triggering it;
b) incubating the mixture obtained in step a), and triggering, in the incubated sample, the generation of thrombin and the formation of a fibrin clot;
c) measuring the time variation of at least one property of the sample obtained in b), in which the fibrin clot forms;
d) establishing the formation profile of the fibrin clot analyzed in c), and extracting from this profile the fibrin formation time (FFT) measured at the inflection point of the tangent of the curve of the profile, and the value of the property (Vp_{(TA)}) of the sample measured at the time to reach (TA) the fibrin polymerization plateau;
e′) calculating the level of D-dimers resulting from intravascular fibrin degradation (R);
e′ 1) by adjusting the level of D-dimers of the sample, Ddi_{S}, as a function of the level of D-dimers generated by inflammation using Vp_{(TA)} determined in d), in order to obtain the level of D-dimers adjusted for inflammation (Ddi_{S/I}); and
e′2) by correcting the level of D-dimers adjusted for inflammation (Ddi_{S/I}) obtained in e′1) for the low D-dimer levels; and
classifying the sample from the patient as a function of inflammation;
f) comparing the level of D-dimers resulting from the degradation of the fibrin R obtained in e), with respect to a threshold, preferably to the threshold of 0.5 μg/ml;
g′) determining the level of D-dimers generated by hyperfibrinolysis (Ddi_{(HF)}) using the level of D-dimers in the sample (Ddi_{S}) and the level R obtained in e′) or using the level of D-dimers adjusted as a function of inflammation, Ddi_{S/I}, obtained in e′1) and the level R obtained in e′); and
h′) comparing the level Ddi_{(HF)} obtained in g′) with respect to a threshold, and comparing the TA/FFT ratio with respect to a threshold, wherein TA is the time to reach the fibrin polymerization plateau obtained in d), and FFT is the fibrin formation time obtained in d).

8. The method according to claim 7, wherein, in step e′), the level R expressed in initial fibrinogen equivalent units (FEUs) is obtained:
e′1) by calculating the level of D-dimers adjusted as a function of inflammation (Ddi_{S/I}) by the following equation:
$${Ddi}_{S / I} = \frac{{Ddi}_{S}}{{[ Fib ]}_{( Vp ( TA ) )}}$$
wherein [Fib]_{(Vp(TA))} is the fibrinogen concentration deduced for the value of the property Vp_{(TA)} on a standard curve having the equation:
y=a ln(x)−b,
wherein:
y is the value of the property measured at the time to reach (TA) the fibrin polymerization plateau,
x is the fibrinogen concentration,
a and b are the constants of the logarithmic equation which links the level of the fibrin plateau and the fibrinogen concentration,
the standard curve having been established for blood samples, the fibrinogen concentration of which has been determined and the value of the property Vp_{(TA)} of which has been determined by steps a)-d);
e′2) by correcting the level Ddi_{S}/1 obtained in e′ 1) by the following equation:
R=Ddi_{S/I}+[0.5−F_{Ddi-S}]
wherein F_{Ddi-S} is a correction factor for the low D-dimer levels (<4 μg/ml), the value of which corresponds to the value of the correction factor for the level of D-dimers of the sample (Ddi_{S}) on the standard curve having the equation:
y=ax²+bx+c
wherein y is the correction factor, F,
x is the level of D-dimers,
a, b and c are the constants of the polynomial equation which links the correction factor and the level of D-dimers,
the standard curve having been established for blood samples, the level of D-dimers of which has been determined and for which the correction factor has been determined empirically so that the level Ddi_{S/I} is related back to the threshold of 0.5 μg/ml FEUs (fibrinogen equivalent units).

9. The method according to claim 7, wherein, in step e′), classifying the sample from the patient as a function of inflammation comprises:
calculating the ratio 1/[Fib]_{(Vp(TA))}, wherein [Fib]_{(Vp(TA))} is the fibrinogen concentration determined in e1′); and
classifying the sample from the patient in group I of patients without inflammation if the ratio 1/[Fib]_{(Vp(TA))}>0.20, or
classifying the sample from the patient in group II of patients with inflammation if the ratio 1/[Fib]_{(Vp(TA))}≤0.20.

10. The method according to claim 7, wherein, in step f), a level R obtained in e′), below the threshold, preferably below the threshold of 0.5 μg/ml, makes it possible to exclude a thrombosis in the patient, and a level R obtained in e′), above the threshold, preferably above the threshold of 0.5 μg/ml, is indicative of the possibility of a thrombosis in the patient.

11. The method according to claim 9, wherein, in step g′), the level of D-dimers generated by hyperfibrinolysis (Ddi_{HF}) is calculated:
g′1) as the ratio between the level R determined in step e′) and the level of D-dimers of the sample, Ddi_{S}, using the following equation:
$${Ddi}_{HF} = {( R / {Ddi}_{S} )}_{patient} = \frac{R}{{Ddi}_{S}}$$
g′2) as the ratio between the level R determined in step e′) and the level Ddi_{S/I}, obtained in e′ 1), using the following equation:
$${Ddi}_{HF} = {( R / {Ddi}_{S / I} )}_{patient} = \frac{R}{{Ddi}_{S / I}} .$$

12. The method according to claim 11, wherein, in step h′):
the level (R/Ddi_{S})ₚₐₜᵢₑₙₜ obtained in g′1) is compared to a threshold:
h′ 1) by determining, for the level Ddi_{S} of the sample, the value of the ratio (R/Ddi_{S}) standard on a standard curve having the equation:
y=ax^{-b}
wherein x is the level of D-dimers,
y is the ratio between the level of D-dimers which result from intravascular fibrin degradation and the level of D-dimers, (R/Ddi_{S}),
a and b are the constants of the equation which links the ratio R/Ddi_{S} and the level of D-dimers,
the standard curve having been established:
using, if the sample from the patient has been classified in group I: blood samples classified in group I by step e′) and the level of D-dimers of which is known or has been determined and the level R of which has been obtained by steps a)-e′2); and
using, if the sample from the patient has been classified in group II: blood samples classified in group II by step e′) and the level of D-dimers of which is known or has been determined and the level R of which has been obtained by steps a)-e′2); and
h″1) by comparing the value of the level of D-dimers generated by hyperfibrinolysis (R/Ddi_{S})ₚₐₜᵢₑₙₜ obtained in g′1) with the value of the ratio (R/Ddi_{S})_{standard} obtained in h′1), wherein:
(R/Ddi_{S})ₚₐₜᵢₑₙₜ less than the ratio (R/Ddi_{S})_{standard} makes it possible to exclude thrombosis in the patient, and (R/Ddi_{S})ₚₐₜᵢₑₙₜ greater than or equal to the ratio (R/Ddi_{S}) is indicative of a possibility of thrombosis in the patient;
the level (R/Ddi_{S/I})ₚₐₜᵢₑₙₜ obtained in g′2) is compared to a threshold:
h′2) by determining, for the level Ddi_{S} of the sample, the value of the ratio (R/Ddi_{S/I})_{standard} on a standard curve having the equation:
y=ax^{-b}
wherein x is the level of D-dimers,
y is the ratio between the level of D-dimers which result from intravascular fibrin degradation and the level of D-dimers adjusted as a function of inflammation, (R/Ddi_{S/I}),
a and b are the constants of the equation which links the ratio R/Ddi_{S/I} and the level of D-dimers,
the standard curve having been established:
using, if the sample from the patient has been classified in group I: blood samples classified in group I by step e′) and the level of D-dimers of which is known or has been determined, the level Ddi_{S/I} of which has been obtained by steps a)-e′1), and the level R of which has been obtained by steps a)-e′2); and
using, if the sample from the patient has been classified in group II: blood samples classified in group II by step e′) and the level of D-dimers of which is known or has been determined, the level Ddi_{S/I} of which has been obtained by steps a)-e′1), and the level R of which has been obtained by steps a)-e′2); and
h″2) by comparing the value of the level of D-dimers generated by hyperfibrinolysis (R/Ddi_{S/I})ₚₐₜᵢₑₙₜ obtained in g′2) with the value of the ratio (R/Ddi_{S/I})_{standard} obtained in h′2), wherein:
(R/Ddi_{S/I})ₚₐₜᵢₑₙₜ less than the ratio (R/Ddi_{S/I})_{standard} makes it possible to exclude thrombosis in the patient, and wherein (R/Ddi_{S/I})ₚₐₜᵢₑₙₜ greater than or equal to the ratio (R/Ddi_{S/I})_{standard} is indicative of a possibility of thrombosis in the patient.

13. The method according to claim 12, wherein, in step h′), comparing the ratio TA/FFT with respect to a threshold comprises:
h′3) calculating the ratio TA/FFT wherein TA is the time to reach the fibrin polymerization plateau determined in step d) and FFT is the fibrin clot formation time determined in step d); and
h″3) if the sample from the patient has been classified in group I:
comparing the ratio TA/FFT with respect to a first threshold, in particular to a first threshold of 1.75, wherein:
a ratio TA/FFT above the first threshold makes it possible to exclude thrombosis and to diagnose thrombophilia or a coagulation activation state in the patient classified in group I, and
a ratio TA/FFT below or equal to the threshold is indicative of a thrombosis in the patient classified in group I;
if the sample from the patient has been classified in group II:
comparing the ratio TA/FFT with respect to a first threshold, in particular a first threshold of 1.65, wherein:
a ratio TA/FFT above the first threshold makes it possible to exclude thrombosis and to diagnose thrombophilia or a coagulation activation state in the patient classified in group II, and
a ratio TA/FFT below or equal to the threshold is indicative of a thrombosis in the patient classified in group II.

14. The method according to claim 13, wherein:
when a thrombosis has been diagnosed in step h″3), the level of D-dimers specific for venous thromboembolism (Ddi_{VTE}) is the level of D-dimers, R, obtained in step e′),
when a thrombosis has been excluded in steps h″1), h″2) and h″3), but the level R obtained in step e′) is above the threshold, preferably above the threshold of 0.5 μg/ml, the method also comprises a step consisting in calculating the level of D-dimers specific for venous thromboembolism (Ddi_{VTE}) using the following equation:
$${Ddi}_{VTE} = 0.5 \times \frac{R}{{Ddi}_{S}}$$
wherein Ddi_{S} is the level of D-dimers in the sample.

15. The method according to claim 1, wherein that the blood sample has a volume of between 1 μl and 300 μl, preferably between 50 μl and 200 μl.

16. The method according to claim 1, wherein that the blood sample is undiluted.

17. The method according to claim 1, wherein the assaying of D-dimers of the sample is carried out according to an immunoturbidimetric or immunoenzymatic method.

18. The method according to claim 1, wherein step a) is carried out by mixing the blood sample from the patient with tissue factor and optionally phospholipids, preferably by mixing the blood sample from the patient with tissue factor and phospholipids.

19. The method according to claim 18, wherein the tissue factor of step a) is present in a concentration of between 0.5 and 5 pM, preferably 2 pM.

20. The method according to claim 18, wherein the mixture of step a) comprises calcium ions.

21. The method according to claim 1, wherein step b) comprises incubating the mixture obtained in step a) for a time of between 20 seconds and 400 seconds, preferably between 60 seconds and 300 seconds, at a temperature between 30° C. and 40° C.

22. The method according to claim 1, wherein, in step b), triggering thrombin generation and fibrin clot formation is carried out by adding calcium ions to the sample incubated.

23. The method according to claim 1, wherein the blood sample from a patient is a plasma sample.

24. The method according to claim 23, wherein the plasma sample is a platelet-poor plasma sample.

25. The method according to claim 23, wherein, in step c), measuring the time variation of at least one property of the sample obtained in b) is carried out by measuring the time variation of the optical density (DOD) at a wavelength of between 350 and 800 nm, preferably at the wavelength of 540 nm.

26. The method according to claim 25, wherein the measurement of the optical density of step c) is carried out at the same wavelength as that used for assaying the D-dimers, 540 nm.

27. The method according to claim 1, wherein the blood sample from a patient is a whole-blood sample.

28. The method according to claim 27, wherein the whole-blood sample is a citrated whole-blood sample.

29. The method according to claim 27, wherein measuring the time variation of at least one property of the sample obtained in b) is carried out by thromboelastography, by rheometry or by image analysis.

30. The method according to claim 1, wherein at least steps c) and d) are carried out on an automated diagnostic device or on a remote biology analyzer, preferably on a coagulation analyzer.

31. An in vitro method for diagnosing venous thromboembolism (VTE) in a patient, comprising steps of:
carrying out an assay of D-dimers specific for VTE in a blood sample from the patient using a method according to claim 1; and
providing a diagnosis regarding the patient.

32. The in vitro method of diagnosis according to claim 31, wherein the diagnosis regarding the patient is (i) exclusion of thrombosis, (ii) acute coagulation activation state, or (iii) thrombosis.

33. The in vitro method of diagnosis according to claim 31, wherein, if the patient is an elderly individual, a patient suffering from cancer, a patient suffering from an infection or a patient suffering from thrombophilia, the assaying of the D-dimers specific for VTE is carried out using a method according to claim 7.

34. The in vitro method of diagnosis according to claim 32, wherein the diagnosis regarding the patient is (i) exclusion of thrombosis, (ii) acute coagulation activation state, (iii) thrombophilia, or (iv) thrombosis.

35. The in vitro method of diagnosis according to claim 34, wherein the diagnosis regarding the patient is a non-serious thrombosis or thrombosis in the case of pulmonary infarction.

36. The method according to claim 6, wherein a high level of D-dimers specific for venous thromboembolism, Ddi_{VTE}, is representative of the extent of the pulmonary embolism or of the deep vein thrombosis.
